(19) United States
(12) Patent Application Publication
(10) Pub. No.: US 2011/0224700 A1
(43) Pub. Date: Sep. 15, 2011

(54) Narrow Profile Surgical Ligation Clip

(75) Inventors: Philip Schmidt, Rougemont, NC (US); Dan Monahan, Raleigh, NC (US); Brad Labarbera, Raleigh, NC (US); Paul Whiting, Wake Forest, NC (US); Steven Morris, Cary, NC (US); David Milton, Garner, NC (US); Thomas Zelmer, Raleigh, NC (US); Cyan Godfrey, Chapel Hill, NC (US)

(73) Assignee: TELEFLEX MEDICAL INCORPORATED, Research Triangle Park, NC (US)

(21) Appl. No.: 13/042,864

(22) Filed: Mar. 8, 2011

Related U.S. Application Data
(60) Provisional application No. 61/312,156, filed on Mar. 9, 2010.

(51) Int. Cl. A61B 17/08

(52) U.S. Cl. 606/151

(57) ABSTRACT

A narrow profile surgical ligation clip has two legs with clamping surfaces joined by a hinge near the proximal ends, allowing the clip to reversibly open and close. A locking mechanism is proximal to the hinge to bias or lock the clip closed, including first and second jaw structures spaced on opposite sides of a longitudinal axis of the clip thereby defining a locking space therebetween. In one embodiment, a wedge or buttress body pivots by application of an external force applied to a proximal end of the clip towards the hinge to move into the locking space such that one or more outer surfaces or projections of portions of the body fit into or abut against complementary surfaces or other parts of the locking mechanism or clip assembly to bias or lock the clip in a closed position and provide additional closing force to the inner clamping surfaces.

### CROSS-REFERENCE TO RELATED APPLICATION

This application claims priority to U.S. provisional patent application No. 61/312,156, filed on Mar. 9, 2010, the disclosure of which is incorporated by reference in its entirety.

### FIELD OF THE INVENTION

The present invention relates to medical devices and in particular surgical clips for ligation of vessels or tissue.

### BACKGROUND

Many surgical procedures require vessels or other fluid ducts or tissue conduits and structures to be ligated during the surgical process, such as, for example, veins or arteries in the human body. For example, many surgical procedures require cutting blood vessels, and these blood vessels may require ligation to reduce bleeding. In some instances, a surgeon may wish to ligate the vessel temporarily to reduce blood flow to the surgical site during the surgical procedure. In other instances a surgeon may wish to permanently ligate a vessel. Ligation of vessels or other tissues can be performed by closing the vessel with a ligating clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, ligating clips are relatively easy and quick to apply. Accordingly, the use of ligating clips in endoscopic as well as open surgical procedures has grown dramatically.

Various types of hemostatic and aneurysm clips are used in surgery for ligating blood vessels or other tissues to stop the flow of blood. Such clips have also been used for interrupting or occluding ducts and vessels in particular surgeries such as sterilization procedures. Typically, a clip is applied to the vessel or other tissue by using a dedicated mechanical instrument commonly referred to as a surgical clip applier, ligating clip applier, or hemostatic clip applier. Generally, the clip is left in place after application to the tissue until hemostasis or occlusion occurs.

Ligating clips can be classified according to their geometric configuration (e.g., symmetric clips or asymmetric clips), and according to the material from which they are manufactured (e.g., metal clips or polymeric clips). Symmetric clips are generally “U” or “V” shaped and thus are substantially symmetrical about a central, longitudinal axis extending between the legs of the clip. Symmetric clips are usually constructed from metals such as stainless steel, titanium, tantalum, or alloys thereof. But, with the advent of high technology diagnostic techniques using computer tomography (CATSCAN) and magnetic resonance imaging (MRI), metallic clips have been found to interfere with the imaging techniques. To overcome such interference limitations, biocompatible polymers have been increasingly used for surgical clips.

Some well known polymeric clips are disclosed in U.S. Pat. No. 4,834,096 and U.S. Pat. No. 5,062,846. These plastic clips generally comprise a pair of curved legs joined at their proximal ends with an integral hinge or heel, and a closure or locking mechanism at their distal ends. Another example of a bio-compatible clip is shown in U.S. Pat. No. 4,671,281, which includes a mechanism to be actuated on a proximal end of the clip for causing the distally extending legs of the clip to converge. However this clip is: (i) rudimentary in construction, (ii) does not provide adequate clip closing or clamping strength, (iii) lacks any complex geometry which would adequately retain the clip in a closed position, and further (iv) is too unstable when closed to be safely applied over vessels. Examples of metal hemostatic clips are shown in U.S. Pat. No. 3,326,216 and U.S. Pat. No. 5,908,430.

In all of the known ligating clips however, there remains a need to improve the effectiveness of clamping about a vessel, while minimizing the damage to the vessel and surrounding tissue. For endoscopic surgical procedures, it is important is use tools and instruments that have the smallest, narrowest profile possible, such as the shafts of a tubular endoscope. Prior art polymeric and metal clips do not lend themselves to deployment through small diameter instrumentation, such as, for example, a ˜5 mm endoscope. Known prior art clips can be very wide profile, especially when in the open position prior to closure and ligation, and thus require larger, wider endoscopic instruments and appliers for use in surgery. It is desirable therefore to provide for a surgical ligation clip that has the narrowest profile possible. It may also be desirable to allow for a clip to be opened again after initial closure, which is especially a problem with known surgical clips, such as metal hemostatic clips. Furthermore, prior art polymeric clips involve locking the distal ends of their legs together in order to clamp down on the vessel or structure being ligated. Such closure of a clip having locking parts at its distal end generally causes or requires dissection, removal, or clearance of additional surrounding tissue, in order to allow the clip's locking features to come together, and/or due to actuation of an applier tool surrounding or applied against the distal clip ends, requiring additional time during a surgical procedure and damage to tissue. In other cases, the user may choose not to prepare a path for the locking features and rely on the locking features penetrating through the tissue. In these cases, the locking feature may have difficulty penetrating the tissue or may have difficulty locking after it has penetrated the tissue. This technique may also result in unintended penetration of tissue or vessels.

Therefore it is desirable to provide a clip which minimizes such dissection of tissue during application. It is further desirable to provide a clip which provides a proper, well-calibrated, reliable clamping force, such that the clip when closed is stable around the vessel ligated.

Accordingly, there is a need to provide an improved surgical ligating clip that serves to reliably secure the tissue or vessel engaged by the clip, while robustly remaining attached to the vessel with a minimum level of damage to tissue.

### SUMMARY OF THE INVENTION

The invention provides, in one or more embodiments, a narrow profile surgical ligation clip which has two legs with clamping surfaces joined by a main clip hinge near the proximal end portion of the clip, allowing the clip to reversibly open and close. One or more embodiments of the surgical clip include a proximal locking mechanism to bias or lock the clip closed, which can be actuated while the legs of the clip are closed. The ligation clip of the present invention can therefore be locked proximally while also being fed through an applier in a closed position. The locking mechanism can include first and second jaw structures extending proximally from the hinge area and spaced on opposite transverse sides of a longitudinal axis of the clip thereby defining a locking space therebetween. In one embodiment, a wedge or buttress body pivots by application of an external force applied to a proximal end of the clip to move said body into the locking space such that one or more outer surfaces or projections of portions of the body fit into or abut against complementary surfaces or other parts of the locking mechanism and/or clip to bias or lock the clip in a closed position and provide additional closing force to the inner clamping surfaces, as well as to stabilize the locked, closed configuration of the clip.

In one or more embodiments of the invention, a surgical ligation clip defines a longitudinal axis and includes first and second legs each extending along the longitudinal axis and having proximal and distal end portions with respect to said longitudinal axis. A clip hinge joins the first and second legs at a point on their respective proximal end portions. The first and second legs each have inner clamping surfaces spanning between the clip hinge and the distal end portions of said first and second legs. The clamping surfaces are apposed when the clip is in a fully closed position. A first jaw structure on the first leg extends proximal to the clip hinge, the first jaw structure having a first curved inner surface extending from the clip hinge and facing the longitudinal axis and being substantially concave viewed from said axis. A second jaw structure on the second leg extends proximal to the clip hinge and has a second curved inner surface extending from the clip hinge. A buttress body extends from and is connected to the second jaw structure by a first living hinge at a proximal end of said second jaw structure. The buttress body has an outer surface on a proximal first end portion thereof on a proximal end of the clip. The first and second jaw structures are spaced from the longitudinal axis on opposite sides thereof and define a locking space therebetween. The buttress body is pivotable about the first living hinge to move into the locking space such that a curved planar segment or segmented abutment portion of the outer surface of the proximal first end portion of the buttress body abuts against the curved inner surface of the first jaw structure to bias the clip in a closed position. In at least one embodiment, the buttress body occupies a majority of a volume of said locking space in said closed position. In one or more embodiments, an overall longitudinal length of the clip is in a range from approximately 0.15 inches to approximately 0.5 inches. The clip can further be made entirely of a polymer material.

In another aspect of the invention, a method of applying a surgical ligation clip includes positioning the clip in an open position proximate an inner anatomical body vessel, the clip having first and second legs each extending along a longitudinal axis of the clip and having proximal and distal end portions with respect to said longitudinal axis, a clip hinge means joining the first and second legs at a point on their respective proximal end portions, the first and second legs each having inner clamping surface means between the clip hinge and the distal end portions of said first and second legs, the clamping surface means being apposed when the clip is in a fully closed position, and a locking means for biasing the legs closed extending proximal to the clip hinge means. An external force is applied substantially along the longitudinal axis to a proximal end portion of one of the legs which forms a portion of the locking means, to move a body formed as a first part of said locking means from a first position to a second position to provide an abutment force between a curved planar segment abutment portion of said body and a curved surface formed on a second part of said locking means disposed on the first leg to bias the clip in a closed position. The method may further include moving the clip through an instrument prior to positioning the clip proximate the vessel, and may also further include that a portion of the instrument opens the clip from a closed position to an open position.

In another aspect, the invention also provides a surgical ligation clip defining a longitudinal axis and including first and second leg means each extending along the longitudinal axis and having proximal and distal end portions with respect to said longitudinal axis. A clip hinge means joins the first and second leg means on respective proximal end portions thereof, the first and second leg means each having inner clamping surface means between the clip hinge means and the distal end portions of said first and second leg means, the clamping surface means being apposed when the clip is in a fully closed position. A locking means biases the legs closed once applied around a vessel, said locking means extending proximal to the clip hinge means. The locking means is capable of actuation by application of an external force substantially along said longitudinal axis to a proximal end of the clip which forms an integral portion of said locking means, to move at least one body disposed on the second leg means as a first part of said locking means from a first position to a second position to provide an abutment force between a curved planar surface segment or segments on said body and a complementary surface formed on a second part of said locking means disposed on the first leg means to bias the clip in a closed position. In one or more embodiments, an overall longitudinal length of the clip is in a range from approximately 0.15 inches to approximately 0.5 inches. The clip can further be made entirely of a polymer material.

In one or more additional embodiments of the invention, a surgical ligation clip assembly includes a surgical clip defining a longitudinal axis and having first and second legs each extending along the longitudinal axis and having proximal and distal end portions with respect to said longitudinal axis. The first and second legs each have inner clamping surfaces spanning between the proximal and distal end portions of said first and second legs, the inner clamping surfaces being opposed and substantially parallel to the longitudinal axis when the clip is in a closed position. A clip hinge joins the first and second leg. A locking ring is disposed in a first position around the clip longitudinally proximal to the clip hinge, prior to application of the clip around a vessel. The clip defines indentations on respective outer surfaces of the first and second legs immediately distal to the clip hinge for receiving the locking ring. After closing the clip, the locking ring can be moved distally past the hinge and positioned over the indentations to lock the clip in a closed position. In one embodiment, the clip hinge joins the second leg at a proximal end of the first leg, the clip hinge being transversely offset from the inner clamping surfaces. In one embodiment, the inner clamping surfaces are disposed between a longitudinal position of the clip hinge and the distal end portions of said first and second legs, and the second leg forms the proximal portion of the clip and defines a transverse shoulder extending from the clip hinge to the inner clamping surface of the second leg, into which the proximal end of the first leg abuts when the clip is closed. The clip may be moved through an instrument prior to positioning the clip proximate the vessel, wherein a portion of the instrument may also open the clip from a closed position to an open position. In one or more embodiments, an overall longitudinal length of the clip can be in a range from approximately 0.15 inches to approximately 0.5 inches. The clip can be made entirely of a polymer material.

In yet another aspect of the invention, a surgical ligation clip, defining a longitudinal axis, includes first and second legs each extending along the longitudinal axis and having proximal and distal end portions with respect to said longitudinal axis. A clip hinge joins the first and second legs at a point on their respective proximal end portions, the first and second legs each having inner clamping surfaces between the clip hinge and the distal end portions of said first and second legs, the clamping surfaces being apposed when the clip is in a fully closed position. A first jaw structure on the first leg extends proximal to the clip hinge, the first jaw structure being angled away from the longitudinal axis. A second jaw structure on the second leg extends proximal to the clip hinge and is angled away from the longitudinal axis. A first proximal heel portion is connected to said first jaw structure by a first living hinge. A second proximal heel portion is connected to said second jaw structure by a second living hinge, the two heel portions being coupled to each other by a third living hinge. The first and second jaw structures are spaced on opposite sides of the longitudinal axis and define a locking space therebetween, the first and second proximal heel portions being pivotable about the first and second living hinges, respectively, to move into the locking space such that the first and second jaw structures are spread apart from the longitudinal axis and bias the clip in a closed position. Complementary interlocking means may be disposed on the respective proximal end outer surfaces of the proximal heel portions.

There has thus been outlined, rather broadly, certain embodiments of the invention in order that the detailed description thereof herein may be better understood, and in order that the present contribution to the art may be better appreciated. There are additional embodiments and features of the invention that will be described below.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

### BRIEF DESCRIPTION OF THE DRAWINGS

FIG. 1 shows a view of a first embodiment of a surgical ligation clip of the present invention;

FIGS. 2a, 2b, and 2c show side, top, and bottom views respectively, of the clip shown in FIG. 1;

FIGS. 3 and 4 show perspective views of the clip shown in FIG. 1 from a first side;

FIG. 5 shows a perspective view of the clip shown in FIG. 1 from the side opposite to that shown in FIGS. 3 and 4;

FIG. 6 is another side view of the clip shown in FIG. 1;

FIG. 6a is a close-up detail view of the portion of the clip shown in FIG. 6 in region “A1” therein;

FIG. 6b is a sectional view of the clip shown in FIG. 6 taken along section B-B in the direction shown in FIG. 6;

FIG. 7 is another side view of the clip shown in FIG. 1 from the opposite side to that shown in FIG. 6;

FIG. 7a is a close-up detail view of the portion of the clip shown in FIG. 7 in region “A2” therein;

FIG. 7b is a sectional view of the clip shown in FIG. 7 taken along section C-C in the direction shown in FIG. 7;

FIGS. 8a, 8b, and 8c, are side, top, and bottom views, respectively, of the clip shown in FIG. 1 in an open position;

FIG. 9 is a perspective view from the bottom of the clip shown in FIG. 8a in the open position;

FIG. 10 is a perspective side view from the top of the clip shown in FIG. 8a in the open position;

FIGS. 11a, 11b, and 11c show side, top, and bottom views respectively, of the clip shown in FIG. 1, with the proximal locking components in locked position;

FIG. 12 is a perspective view from the top of the clip shown in FIG. 11a;

FIG. 13 is a side view of the clip shown in FIG. 11a;

FIG. 13a is a close-up detail view of the portion of the clip shown in FIG. 13 in region “A3” therein;

FIG. 14 is a side view of the clip shown in FIG. 11a from the side opposite to that shown in FIG. 13;

FIG. 14a is a close-up detail view of the portion of the clip shown in FIG. 14 in region “A4” therein;

FIG. 15 is a view of the clip shown in FIG. 1;

FIG. 15a is a close-up detail view of the portion of the clip shown in FIG. 15 in region “A5” therein;

FIG. 16 is a side view of a surgical ligation clip according to another embodiment of the invention;

FIG. 16a is side view of the clip shown in FIG. 16, with the proximal locking components in locked position;

FIG. 17 is a side perspective view of the clip shown in FIG. 16, taken from the side opposite to that shown in FIG. 16;

FIG. 18 is another side view of the clip shown in FIG. 16;

FIG. 18a is a view showing the proximal end of the clip shown in FIG. 18;

FIG. 19 is a first side view of the surgical ligation clip of FIG. 1 applied over a vessel in a closed and locked position;

FIG. 20 is a second side view of the surgical ligation clip of FIG. 1 applied over a vessel in a closed and locked position;

FIG. 21a is a side view of a surgical ligation clip according to another embodiment of the invention;

FIG. 21b is an exploded side view of the surgical ligation clip of FIG. 21a;

FIG. 22a is a side perspective view of the clip shown in FIG. 21a;

FIG. 22b is an exploded side perspective view of the surgical ligation clip of FIG. 21a;

FIG. 23a is another side perspective view of the clip shown in FIG. 21a;

FIG. 23b is another exploded side perspective view of the surgical ligation clip of FIG. 21a;

FIG. 24a is a side view of the clip in FIG. 21a in a legs-open position;

FIG. 24b is a side view of the clip in FIG. 21a in a closed and locked position;

FIG. 25a is a side perspective view of the clip shown in FIG. 21a in a legs-open position;

FIG. 25b is a side perspective view of the clip shown in FIG. 21a in a closed and locked position;

FIG. 26a is a side view of a surgical ligation clip assembly according to another embodiment of the invention;

FIG. 26b is a side view of the clip assembly of FIG. 26a in an assembled, legs-closed and unlocked position;

FIGS. 26c and 26d are side perspective views of the clip assembly shown in FIGS. 26a and 26b, respectively;

FIG. 27a is a side view of the clip assembly of FIG. 26a in an assembled, legs-open and unlocked position;

FIG. 27b is a side view of the clip assembly of FIG. 26a in an assembled, legs-closed and locked position;

FIGS. 27c and 27d are side perspective views of the clip assembly shown in FIGS. 27a and 27b, respectively;

FIG. 28 is a side view of a surgical ligation clip according to another embodiment of the invention;

FIG. 28a is a side view of the clip shown in FIG. 28 with the legs in an open position;

FIG. 29 is a rear side perspective view of the clip shown in FIG. 28, with the proximal locking components in an unlocked position;

FIG. 30 is a rear side perspective view of the clip shown in FIG. 28, with the proximal locking components a locked position;

FIG. 30a is a side view of the clip shown in FIG. 30 in the locked position.

### DETAILED DESCRIPTION

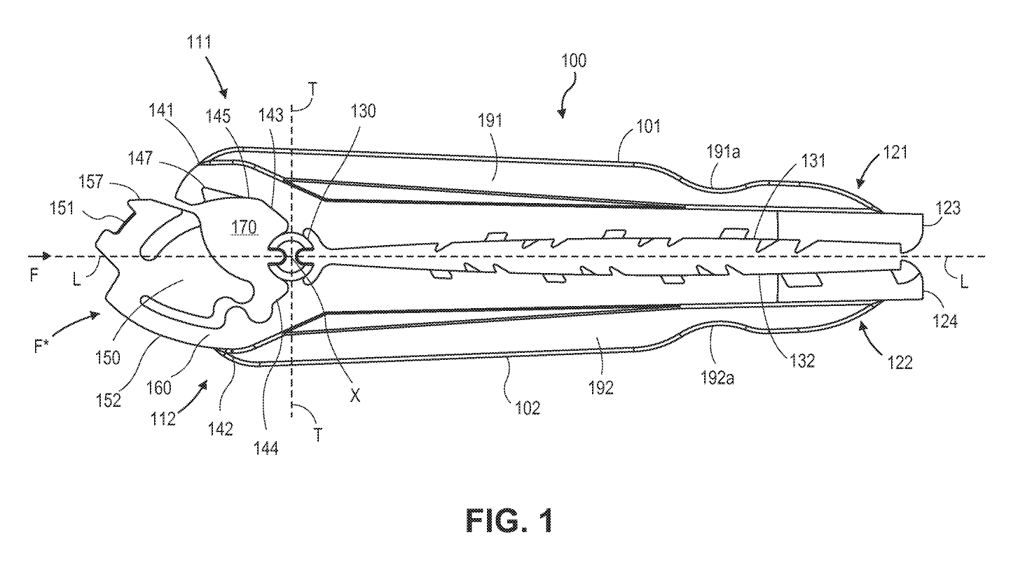

The invention will now be described with reference to the drawing figures, in which like parts are referred to with like reference numerals throughout. FIG. 1 shows a view of a first embodiment of a surgical ligation clip 100 of the present invention. The clip 100 defines a longitudinal axis “L” along its longest dimension and includes a first leg 101 and a second leg 102 each extending along the longitudinal axis L and having proximal 111, 112 and distal 121, 122 end portions with respect to said longitudinal axis. As used herein, the term “proximal” shall refer to the portion of the clip referenced herein which is away from the tips of the clip which open, and “distal” shall refer to the portion of the clip at the tips which open, in accordance with the convention that the clip is inserted distal tip first through an instrument towards an anatomical body to be ligated, such that distal generally refers to the direction away from the user or applier of the surgical clip and proximal refers to the direction opposite to distal.

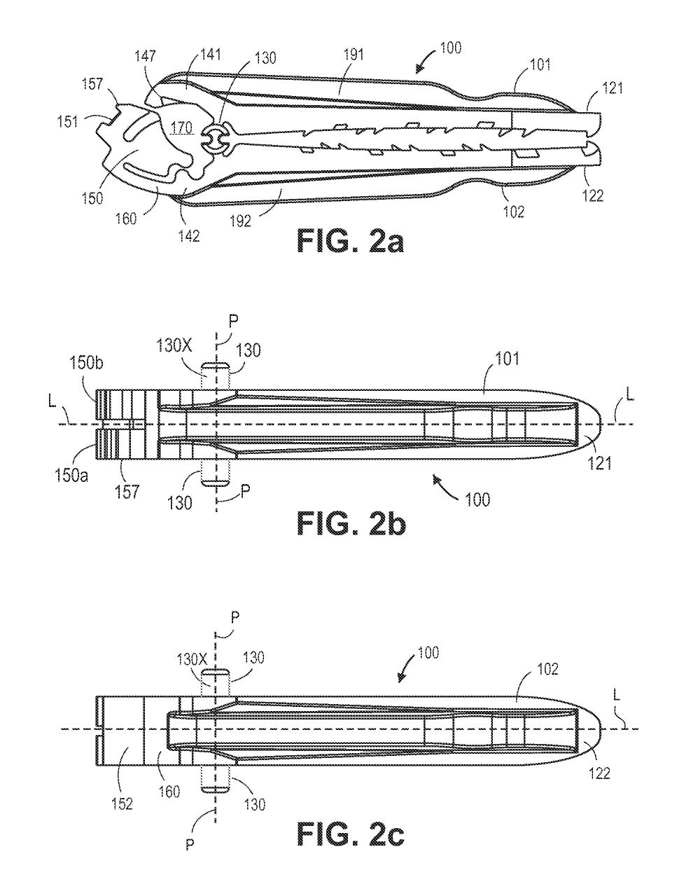

In clip 100, a clip hinge 130 joins the first and second legs 101, 102 at a point on their respective proximal end portions 111, 112, the first and second legs each having respective inner clamping surfaces 131, 132 between the clip hinge 130 and the distal ends 123, 124 of said first and second legs, the clamping surfaces being apposed when the clip is in a fully closed position. As used herein, the term “apposed” when used with regard to the inner clamping surfaces 131, 132 shall mean close to, or nearly in contact with each other, allowing for some small spacing therebetween or a concave radius of curvature for the clamping surfaces, such to allow for a clipped vessel to reside between such apposed surfaces, as is more fully illustrated herein and with respect to the drawing figures. The clip hinge 130 can include a bar or cylindrically shaped body or tube which defines a lateral pivot axis “P” (shown in FIGS. 2b and 2c) about which the legs 101 and 102 pivot as the clip moves from open to closed position and vice versa. A first jaw structure 141 on the first leg 101 extends proximal to a transverse axis “T” which is perpendicular to both the longitudinal axis L and lateral pivot axis P, all intersecting at a point “X” centered on the clip hinge 130, as shown in FIG. 1. As used throughout herein, the term “lateral” shall directionally mean orthogonal to both the directions of the longitudinal axis L and transverse axis T, and parallel to pivot axis P as shown in the figures. The first jaw structure 141 includes a first curved inner surface 143 extending from the clip hinge 130, the first curved inner surface 143 having a complex surface which is oriented at changing angles with respect to, but is generally facing towards, the longitudinal axis L, as shown in FIG. 1. The curved inner surface 143 is therefore substantially concave when viewed from the longitudinal axis (or plane spanning the longitudinal axis and pivot axis). As used herein, the term “substantially concave” shall mean a surface which is concave in overall curvature, but which may include one or more component areas which may have convex segments or protrusions, such as a notch surface or recess for mating thereto. A second jaw structure 142 is on the second leg 102 extending proximal to the transverse axis T and has a second curved inner surface 144 extending from the clip hinge 130. As used herein, the “curved inner surface” can include either a single smoothly curved surface segment, or a series of connected curved or straight planar segments, which, taken together, form an overall generally curving surface. As described herein, the surgical clip of the present invention provides that the jaws 141 and 142 are each substantially proximal to a transverse plane extending through transverse axis T and lateral pivot axis P, thus behind the clip hinge 130, thereby providing a means for actuating the clip legs 101 and 102 and biasing or locking the clip and its mating faces 131, 132 in a closed position, which biasing or locking means can be actuated and/or applied by acting only on the proximal end portions of the clip 100, without having to lock the distal ends 123, 124 to each other or use a clip applier tool which acts on said distal ends 123, 124, thereby obviating the need to dissect tissue around the distal end of the clip as in previously known surgical ligation clips.

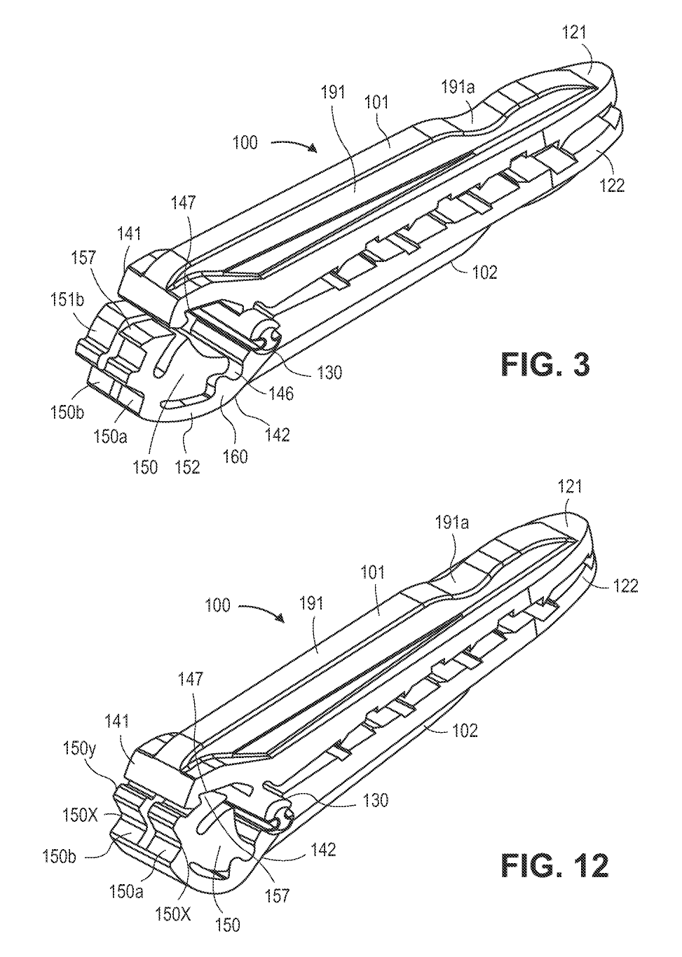

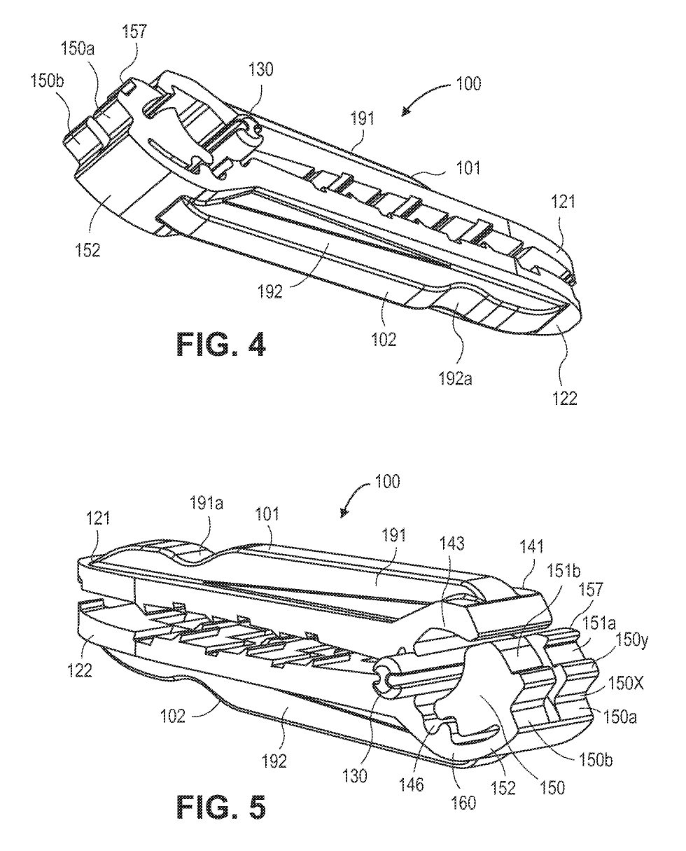

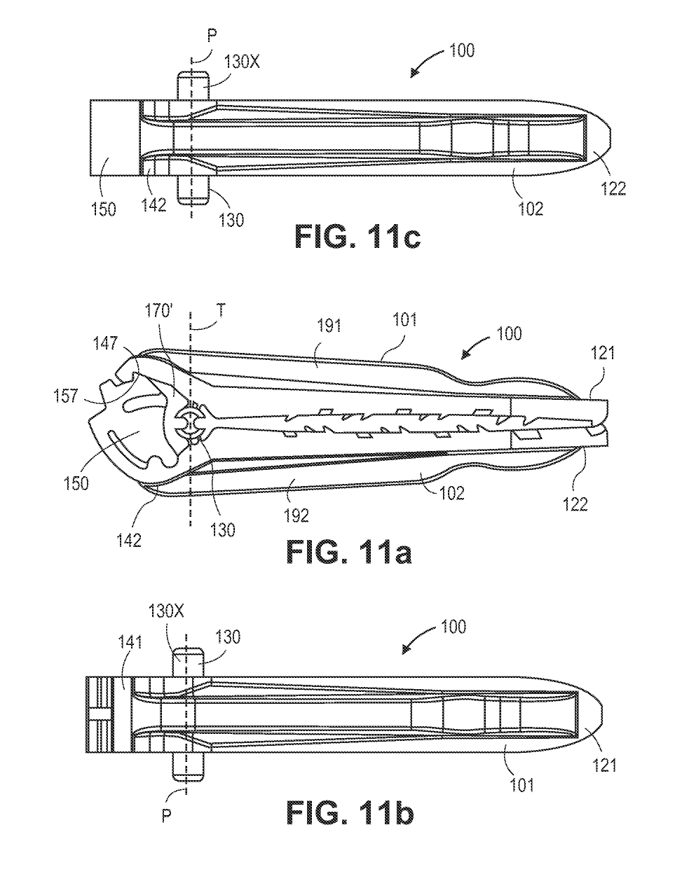

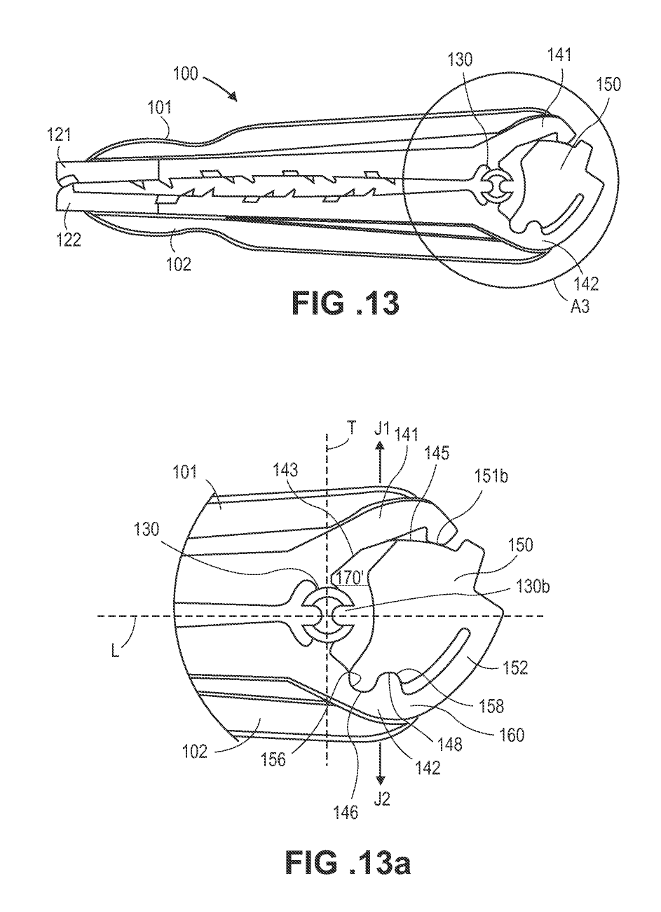

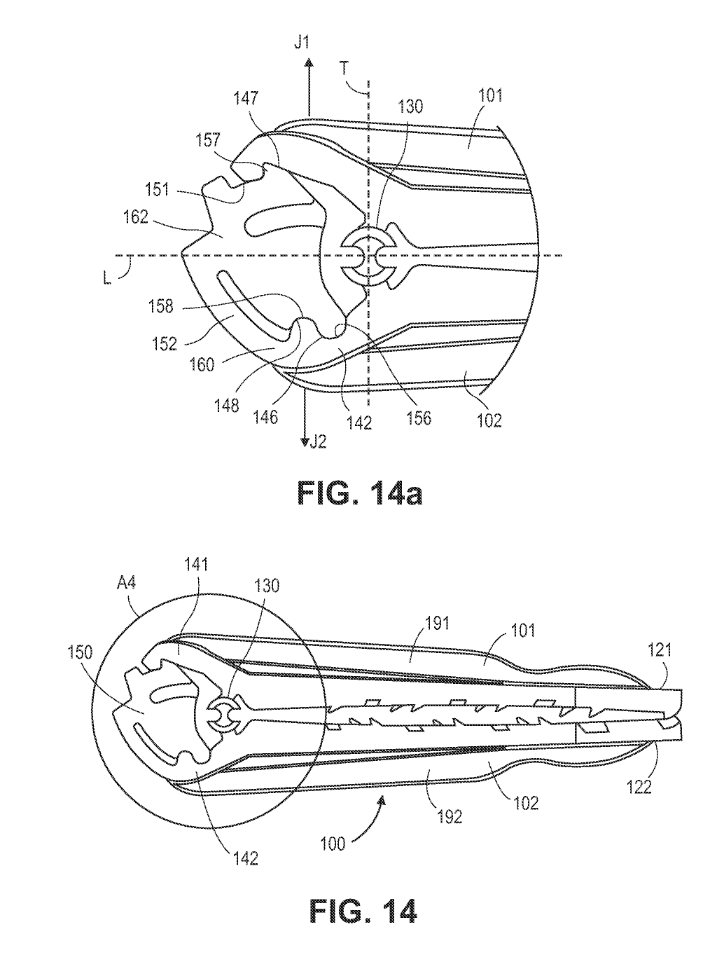

As shown in FIG. 1, the means for biasing or locking the clip closed includes a wedge or buttress body 150 which extends from and is connected to the second jaw structure 142 by a first living hinge 160 at a proximal end of said second jaw structure 142, the buttress body 150 having an outer surface 151 at a proximal first end portion thereof, which is also disposed approximately as the proximal end of the clip 100 overall. The first and second jaw structures 141, 142 are spaced on opposite sides of the longitudinal axis L and define a locking space 170 therebetween. The wedge or buttress body 150 is pivotable about the living hinge 160 to move into the locking space 170 such that the outer surface 151 of the proximal first end portion of the buttress body 150 abuts against a proximal portion 145 of the curved inner surface 143 of the first jaw structure 141 to bias the clip in a closed position (as best shown in FIGS. 11a, and 12-14). Although the clip 100 is shown in FIG. 1 in a closed position, this is with the locking means of the first and second jaws 141, 142 and buttress body 150 being in the “unlocked” position as shown in FIGS. 1, 2a, and 3-7. Once the buttress body is in the “locked” position as shown in FIGS. 11a and 12-14, the first and second jaws 141, 142 are urged or spread apart (shown, as an example, by arrows “J1” and “J2” in FIGS. 13a and 14a) by action of surfaces of the wedge/buttress body 150 acting on portions of curved inner surfaces 143, 144, which act as moments about the clip hinge 130 and lateral pivot axis P to urge the legs 101, 102 and its inner clamping surfaces 131, 132 to become more closely apposed to each other, thereby providing additional clamping and closing force over a vessel around which the clip is applied.

A variety of means may be used to actuate the wedge or buttress body 150 from the unlocked position in FIG. 1 to the locked position shown in FIGS. 11a, 12-14. As shown in FIG. 1, an external force, shown, for example, as arrow “F” in FIG. 1, may be applied to a proximal end of the pivoting buttress body 150, in this example the external force F being substantially aligned with the longitudinal axis L. Alternatively, the external force applied may be at a small angle to the longitudinal axis L, such as, for example, a force shown by arrow “F*” shown in FIG. 1. In either case, the applied external force will create a moment about living hinge 160 to pivot the buttress body 150 into the locking space 170. The external force may be applied by an actuating rod or other structural means in an applier instrument, or may be another clip as fed through a multi-clip applier. As one example, the clip 100 may be inserted through an instrument having a bore or channel for receiving the clip 100, through which the clip 100 may travel distally for positioning near a vessel during a surgical procedure. The clip may be inserted in a legs closed position, but with the proximal locking means including buttress body 150 in open, unlocked position. Because the clip 100 can be inserted in such fashion in closed form, the clip forms a narrow profile and can fit in smaller sized surgical instruments, thereby allowing for smaller incisions and tissue dissection or damage during surgery. A rod or other actuating mechanism translating or moveable on the instrument inserting the clip, or a second instrument or second clip used in conjunction with the instrument used for inserting and positioning the clip in place, maybe used to lock the clip by application of an external force on the proximal end portion of the clip as discussed above.

Thus, a method of applying a surgical ligation clip on a vessel in accordance with an embodiment of the invention includes positioning a clip, such as, for example, clip 100, in an open position proximate a vessel, the clip having first and second legs each extending along a longitudinal axis of the clip and having proximal and distal end portions with respect to said longitudinal axis, a clip hinge means joining the first and second legs at a point on their respective proximal end portions, the first and second legs each having inner clamping surface means between the clip hinge and the distal end portions of said first and second legs, the clamping surface means being apposed when the clip is in a fully closed position. A locking means for biasing the legs closed may extend proximal to a transverse axis perpendicular to the longitudinal axis intersecting at a point centered on the clip hinge. The method includes applying an external force to a proximal end portion of the clip or of one of the legs which forms a portion of the locking means, to move a body formed as a first part of said locking means from a first position to a second position to provide an abutment force between said body and a surface formed on a second part of said locking means to bias the clip in a closed position. In the method, an instrument may be used, wherein, in moving the clip through the instrument prior to positioning the clip proximate a vessel, a portion of the instrument opens the clip from a closed position to an open position, such that the legs of the clip open for placement of the clip around a vessel. The locking means may then be applied to the proximal end portion of the clip to move and bias the legs closed and clamp the clip more fully over the vessel.

In FIG. 1, the clamping surfaces appear substantially parallel to each other, oriented, in the clip closed position, substantially or very close to parallel to a plane extending through the longitudinal axis L and lateral pivot axis P. However, in an embodiment of the invention, the inner clamping surfaces 131, 132 may be slightly curved concave when facing said surfaces, such that the surfaces bow away from the longitudinal axis L and straighten slightly when clamping force is applied by action of the locking mechanism of the buttress body 150 acting against jaws 141, 142. This allows for enhanced grasping and occlusion of vessels around which the clip 100 is applied, wherein the clamping force is spread more evenly across the clamping surface.

The living hinge 160 connecting the wedge or buttress body 150 to the second jaw 142 can be integral to the second jaw 142 such that the clip body of second leg 102 proximal to transverse axis T extends as a single unitary structure including the second jaw 142 and entire wedge or buttress body 150. Accordingly, in the wedge or buttress body 150, a lateral beam or curved body 152 connects the living hinge 160 to the rest of the buttress body 150, which beam 152 curves from the living hinge 160 (which is separated by a distance from the longitudinal axis L) towards the longitudinal axis L. As shown in FIG. 1 portions of wedge of buttress body 150 can be oriented on both sides of longitudinal axis L. The pivot axis of living hinge 160 extends in a lateral direction parallel the lateral pivot axis P of the main clip hinge 130.

The present invention provides, in various embodiments, a locking mechanism cooperating between the buttress body 150 and another portion of the clip. In the clip 100 shown in FIG. 1, the proximal end portion 145 of the curved inner surface 143 of the first jaw structure 141 defines a notch 147 recessed from said curved inner surface 143, and the buttress body 150 defines a detent 157 formed on the outer surface thereof, the detent 157 mating with the notch 147 when the buttress body 150 is pivoted into the locking space 170 to bias the clip in the closed position, as best shown in FIGS. 11a, 12, and 14.

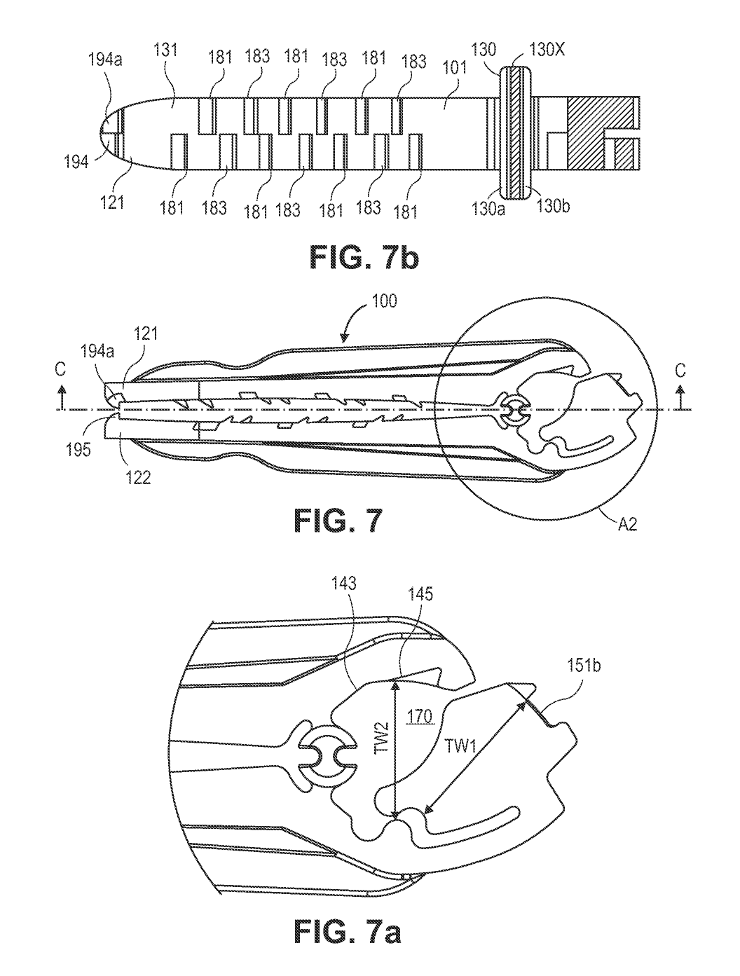

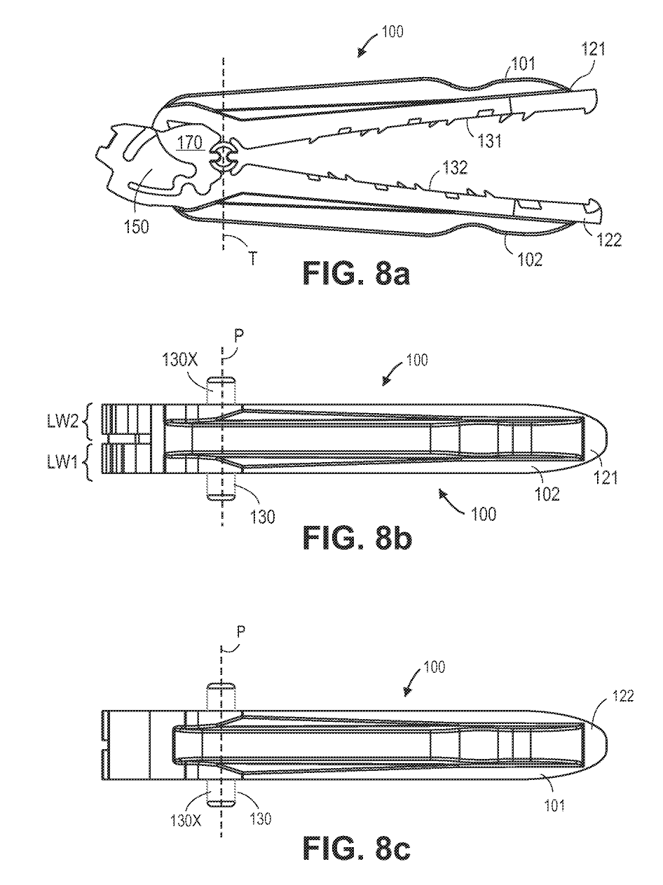

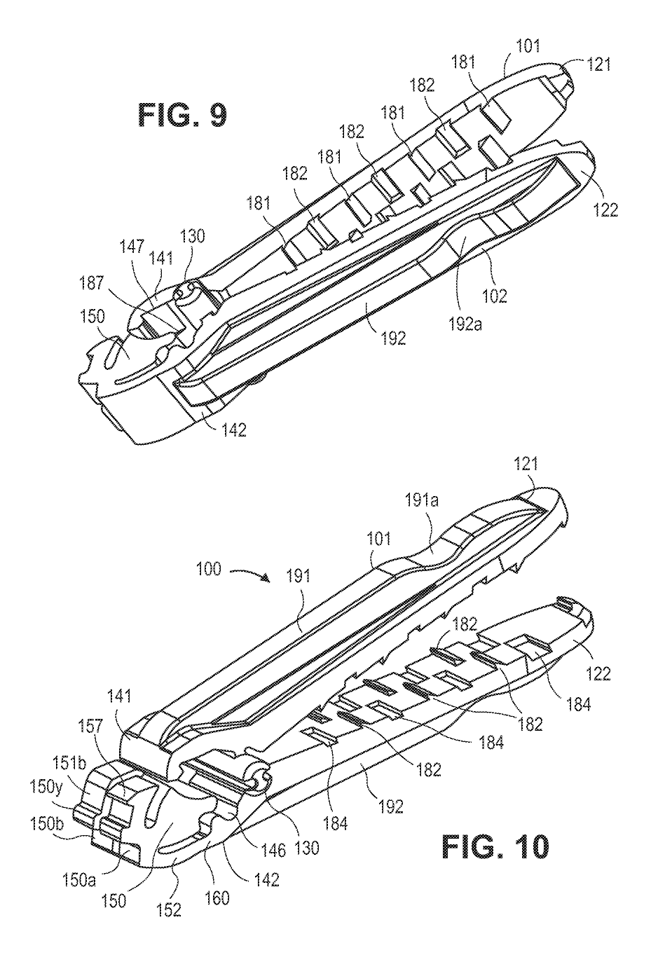

FIGS. 2a, 2b, and 2c show side, top, and bottom views respectively, of the clip shown in FIG. 1. As shown in FIG. 2b, the wedge or buttress body 150 can be divided into two lateral sections or portions 150a and 150b, each on opposite sides of the longitudinal axis L as shown, and can form approximate lateral halves of the buttress body 150, with a possible space or small channel in-between. Lateral portion 150b of the buttress body 150 can have a width in a plane spanning the transverse and longitudinal axes sufficient to exceed a complementary width formed by the locking space 170 to create an interference fit between the proximal end portion 145 of the curved inner surface 143 of the first jaw structure 141 and the outer surfaces 151a, 151b on the proximal first end portion outer surface 151 of the buttress body 150, to bias the clip in a closed position. An example of the transverse width of said lateral portion 150b is shown as distance “TW1” in FIG. 7a, with complementary width “TW2” being formed by the locking space 170, it being understood that TW1 is slightly greater than TW2 in order to create the interference fit. In the embodiment as shown in FIGS. 1, 2b, and 7a, on lateral portion 150b there is no detent 157, and said lateral portion 150b of the buttress body is formed by a partial lateral width of the buttress body 150. Thus, as shown in FIG. 2b, the notch 147 and detent 157 are formed on corresponding partial lateral sections or slices of the buttress body 150 and first jaw structure 141, respectively, this lateral section 150a of buttress body 150 being on the opposite side thereof to the lateral section 150b. In this manner, the buttress body 150, once locked into place as shown in FIG. 12, is prevented from moving laterally from side to side since the notch 147 and detent 157 interlock only extends laterally partially across the clip, the detent 157 being limited in lateral movement by a shoulder 187 formed by a termination of the notch 147 laterally into the first jaw structure 141, as shown in FIG. 9. As shown in FIG. 8b, the lateral slice of buttress body 150 only extends for a lateral width LW1 which includes detent 157, which the lateral slice LW2 of buttress body 150 on the other side of the clip does not include the detent 157. In this manner, the proximal locking mechanism of the clip 100 is more stable in lateral directions, which is also useful for keeping all parts of the clip together in the event the living hinge 160 may break.

As best shown on FIG. 5, the outer surface 151 on proximal first end portion of buttress body 150 on a proximal end of the clip 100 defines one or more surfaces which form a curved planar segment abutment portion, which in the embodiment as shown includes curved planar segment abutment portions 151a and 151b. As used herein, the “curved planar segment abutment portion” formed by a surface may include a single curved surface segment or a series of curved or straight planar surface segments connected to one another which form an overall generally curved surface, each of the surface segments extending as a surface at least laterally. In the embodiment shown in FIG. 5, curved planar segment abutment portion 151a included planar and curved surface segments formed by the notch 157 and extends laterally for about one-half of the lateral width of clip 100, curved planar segment abutment portion 151b includes planar and curved surface segments which also extend laterally for about one-half of the lateral width of clip 100. Each of the curved planar segment abutment portions 151a and 151b on outer surface 151 forms a substantial abutment surface that pushes against complementary curved inner surfaces of jaw 141 to provide a stronger and more stable locking mechanism for clip 100. This is provided, at least in part, by the relatively larger and wider surface areas, lateral spans, and segmented surfaces with interlock and abut against eachother to provide enhanced holding strength and stability, beyond what has been previously known or practiced in the field of surgical ligation clips.

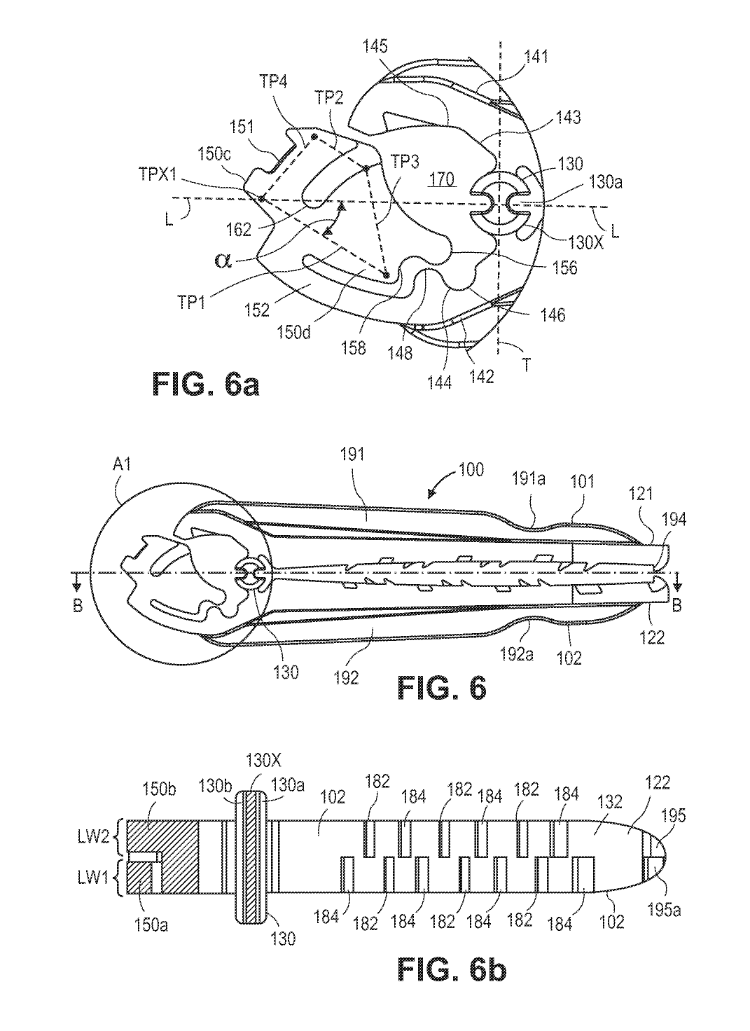

As best shown in FIG. 6a, the second curved inner surface 144 on the second jaw structure 142 forms a first laterally spanning recessed groove 146 separated from the clip hinge 130 and a first laterally spanning ball-shaped or rounded protruding surface 148 proximal to said recessed groove 146, and a distal second end portion of the buttress body 150 forms a second laterally spanning recessed groove 158 and a second laterally spanning ball-shaped or rounded protruding surface 156 distal to said second recessed groove which are shaped complementary to the first rounded surface 148 and first recessed groove 146, respectively, so as to mate in abutment when the buttress body 150 is pivoted into the locking space 170 to further stabilize and bias the clip in a closed position. The first recessed groove 146, first rounded surface 148, second recessed groove 158, and second rounded surface 156 may extend laterally all the way across the lateral width of the buttress body 150, such that the first rounded surface 148 and second rounded surface 156 are not spherically shaped but rather form an extended, laterally-spanning, rounded, semi-cylindrical surface which can mate in corresponding semi-cylindrical shaped grooves formed by first recessed groove 146 and second recessed groove 158.

As shown in FIG. 6a, the buttress body 150 can further define a second living hinge 162 extending laterally between the proximal first end portion 150c of buttress body 150 and a distal second end portion 150d, wherein the proximal first end portion 150c including outer surface 151 further pivots about said second living hinge 162 when the buttress body 150 moves into the locking space 170, allowing the outer surface 151 of the proximal first end portion 150c of the buttress body to flex towards the longitudinal axis L prior to abutment against the curved inner surface 143 of the first jaw structure 141.

As best shown in FIGS. 5 and 12, the outer surface of the proximal end of the buttress body 150, or clip 100 itself, defines a V- or L-shaped laterally spanning notch 150x on the proximal end of the clip 100 and further defines a laterally spanning flange 150 y extending from said notch 150x adjacent to the curved planar segment abutment portions 151a and 151b. Each of notch 150x and flange 150y may be divided into two lateral sections or components divided by a small space or channel therebetween as they are disposed on the lateral sectional halves 150a and 150b of the buttress body 150. The notch 150x provides a receiving space for the tip of an instrument, pushing or actuating rod, or another clip, so as to enable a more stable actuation of the buttress body 150 into locking space 170 to lock the clip 100. The flange 150y may act to limit the movement of buttress body 150 once fully inserted into locked position inside space 170, and also further stabilizes the locking mechanism for the clip 100.

In the embodiment shown in FIGS. 1-15, the buttress body may occupy a majority of a volume defined by locking space 170 when it is moved into clip locked position so as to bias the legs 101, 102 in a closed position. The volume defined by the locking space is limited by the lateral width of the clip legs 101, 102 near the hinge 130 and the jaws 141 and 142. As shown in FIG. 13a, the remaining locking space 170′ between jaws 141 and 142, once the clip is locked by movement of the buttress body 150 into space 170, is less than half the volume of the locking space 170 as shown in FIG. 6a. The presence of a bulky body like buttress body 150 which occupies the majority of the volume or space between proximal extending jaws 141 and 142 when the clip 100 is in the locked position further provides a greater strength and stability to the locking of said clip.

In the embodiment shown in FIGS. 1-15, and as shown in FIG. 6a, the buttress body 150 can be characterized in one way as having a core mass which has, in a transverse plane spanning the longitudinal and transverse axes, a cross-section which approximately spans a trapezoidal shape, having rounded curved sides extending from the sides TP1, TP2, TP3, TP4 of the trapezoid. Side TP1 defines the longest side and one of the parallel sides of the trapezoid, while side TP2 defines the shorter parallel side. Side TP3 defines the longer and more distal of the non-parallel sides, while side TP4 defines the shorter and more proximal non-parallel side. Side TP1 is therefore connected to sides TP3 and TP4. When the clip is in the unlocked position as shown in FIG. 6a, and the buttress body 150 is fully extended away from the clip hinge 130 out in the most proximal position, the vertex TPX1 of sides TP1 and TP4 lies approximately on or near the longitudinal axis L, and side TP1 makes an angle α below the longitudinal axis, towards proximal jaw 142, such angle α being, in one embodiment, approximately 30 degrees. As shown in FIG. 6a, the rounded laterally-spanning protuberance 156 extends substantially from side TP3.

The clip hinge 130 can also be a resilient hinge providing additional biasing force to maintain the inner clamping surfaces 131, 132 of the legs towards a closed position. A span of each leg extending from the clip hinge 130 to its respective distal tip 123, 124, can be, in one embodiment of the present invention, at least 75% to 80% of an overall length of the clip. As shown in FIGS. 2b and 2c, the clip hinge 130 can define lateral bosses which extend laterally from the side surfaces of the clip legs, defining a bossed width or span which is greater than the clip width.

In the embodiment shown in FIGS. 1-15, the clip hinge 130 is formed as a laterally extending bar 130x integrally formed with the first and second legs 101, 102, each leg being resiliently coupled to first and second transverse sides of said bar, the bar 130x further defining laterally spanning grooves 130a and 130b on longitudinally distal and proximal sides of the bar, respectively. These grooves 130a and 130b further enable the clip 100 to flex as pivoting about the lateral axis of hinge 130, and further provide a resilient pivoting moment or force about said hinge.

Furthermore, in the embodiment shown in FIGS. 1-15, flanges 191 and 192 extend longitudinally across respective outer surfaces of each of the first and second legs 101, 102 which are on opposite sides to the inner clamping surfaces 131, 132 of each respective leg, the flange 191 of the first leg 101 extending from the first jaw structure 141 to the distal end portion 121 of the first leg 101, the flange 192 of the second leg 102 extending from the second jaw structure 142 to the distal end portion 122 of the second leg 102. Each of the flanges 191, 192 defines a transverse indentation 191a, 192a proximate the distal end portions 121, 122 of the legs 101, 102. The flanges 191 and 192 provide a rigidity to legs 101 and 102, respectively, such that said legs do not easily bend. Transverse indentations 191a and 192a provide a means for a clip applier to better actuate or grip the legs 101, 102.

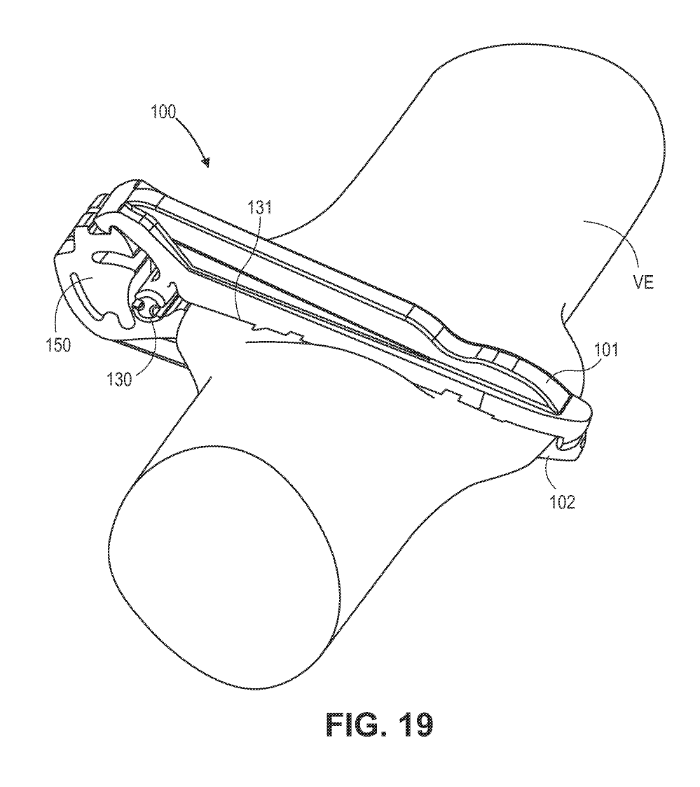

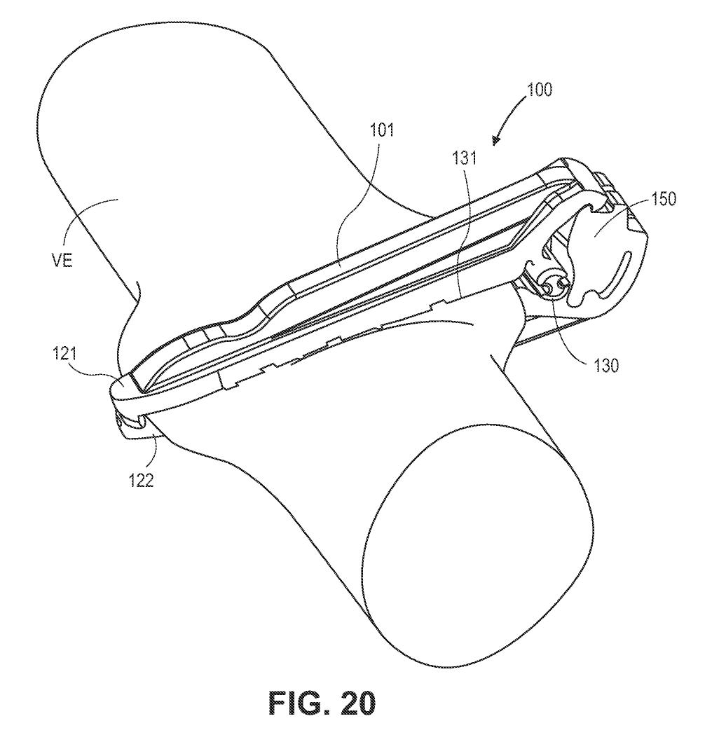

The clip 100 further includes serrations, ridges, or teeth 181, 182 on the inner clamping surfaces 131 and 132, respectively, as shown in FIGS. 6b and 7b, and 9, 10, and 15a. The teeth or ridges 181, 182 provide additional grasping means to better attach and clamp the clip 100 onto a vessel when closed. The teeth or ridges 181, 182 are disposed to fit into complementarily arranged grooves 183 and 184 on the clamping surfaces 131 and 132, respectively. The teeth 181, 182 may have a slanted orientation, extending proximally, so as to better grip tissue. As best shown in FIGS. 6-6a and 7-7a, a pair of distal hook elements 194 and 195 may be disposed on the absolute distal tips of legs 101 and 102, respectively, each hook 194 and 195 offset laterally with respect to eachother to form a scissor-like configuration, such that each hook 194 and 195 fit into corresponding recesses 195a and 194a, respectively, on the distal tips of legs 102 and 101, respectively. This mechanism provides means to further grip and contain tissue with the space between the clamping surfaces 131, 132 when the clip 100 is applied to body vessel, as illustrated in FIGS. 19 and 20.

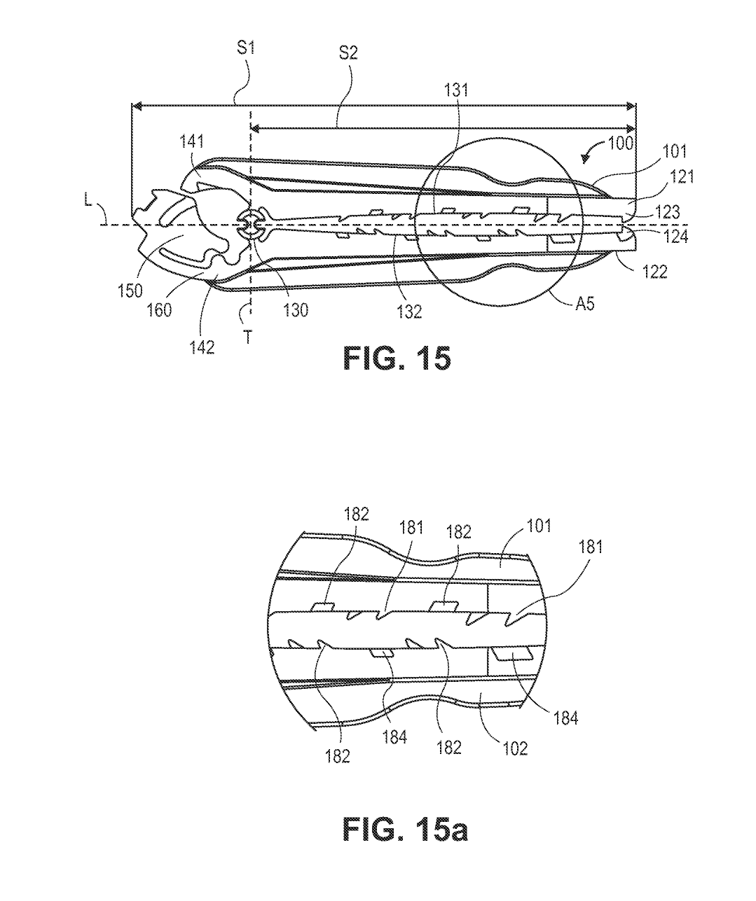

The clip 100 may be in a range of sizes. As shown in FIG. 15, an overall length “S1” of the clip 100 may be approximately 0.50 inches; the length “S2”, between the intersection of transverse axis T and longitudinal axis L centered at clip hinge 130 and the distal tip of the clip, may be approximately 0.40 inches, and the radius of curvature of the inner mating or clamping surfaces 131, 132 of the legs 101, 102 may be approximately 3.0 inches. Such sizes and dimensions are given as an example, and it is understood that the clip may, in one or more embodiments of the invention, vary in size ranging from approximately 0.15 to 0.80 inches in overall longitudinal length, and from approximately 0.03 to 0.15 inches in lateral width. As one embodiment of the invention, the illustration of clip 100 in FIG. 15 is shown as a scaled magnification of actual size, and shows all the parts of the clip 100 in actual proportion to each other.

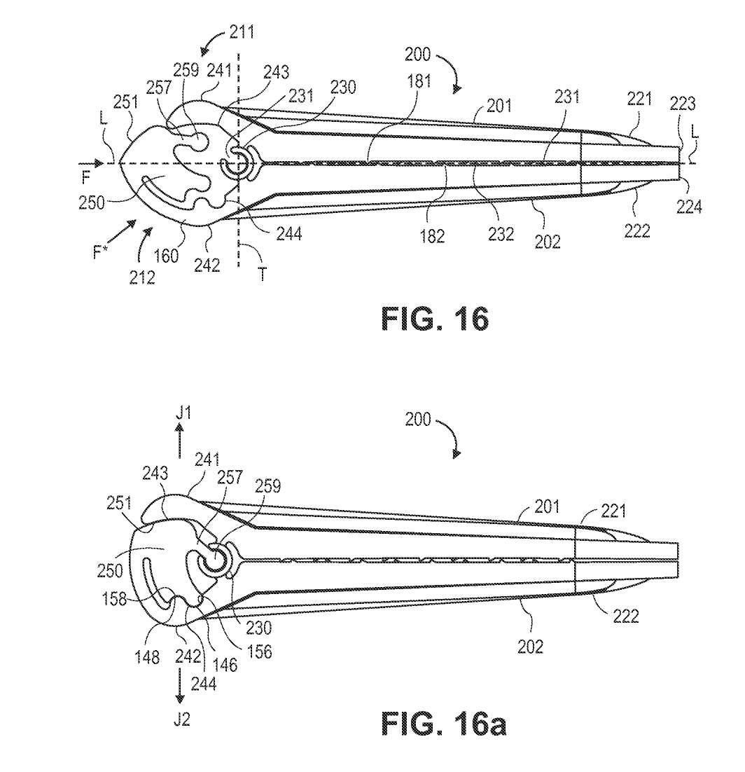

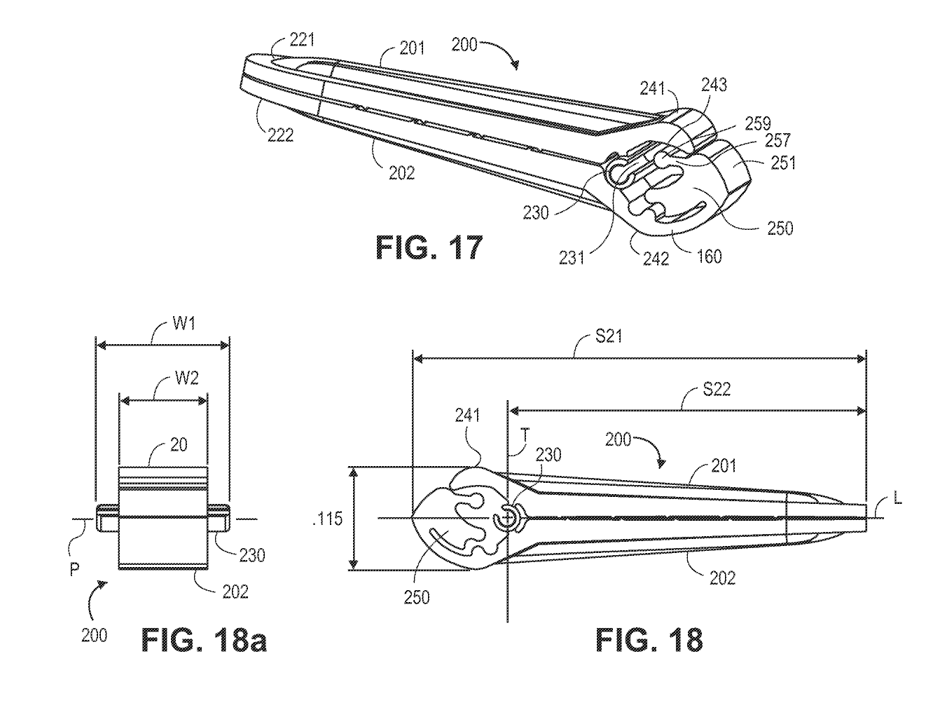

FIG. 16 is a side view of a surgical ligation clip according to another embodiment of the invention. In FIG. 16, a surgical ligation clip 200 defines a longitudinal axis “L” and includes first and second legs 201, 202 each extending along the longitudinal axis and having proximal 211, 212 and distal 221, 222 end portions with respect to said longitudinal axis. A clip hinge 230 joins the first and second legs 201, 202 at a point on their respective proximal end portions, the first and second legs 201, 202 each having inner clamping surfaces 231, 232 between the clip hinge 230 and the distal ends 223, 224 of said first and second legs, the clamping surfaces being apposed when the clip is in a fully closed position. The clip hinge 230 can include a bar or cylindrically shaped body or tube which defines a lateral pivot axis “P” (shown in FIG. 18a) about which the legs 201 and 202 pivot as the clip moves from open to closed position and vice versa. A first jaw structure 241 on the first leg 201 extends proximal to a transverse axis T perpendicular to the longitudinal axis L intersecting at a point centered on the clip hinge 230. The first jaw structure 241 includes a first curved inner surface 243 extending from the clip hinge 230 and facing the longitudinal axis L. A second jaw structure 242 on the second leg 202 extends proximal to the transverse axis T and includes a second curved inner surface 244 extending from the clip hinge 230. A wedge or buttress body 250 extends from and is connected to the second jaw structure 242 by a first living hinge 160 at a proximal end of said second jaw structure, the buttress body 250 having an outer surface 251 at a proximal first end portion thereof. The first and second jaw structures (241, 242) are spaced on opposite sides of the longitudinal axis L and define a locking space 170 therebetween. Clip 200 is similar to clip 100, except that wedge or buttress body 250 further includes, unlike body 150 on clip 100, a projection 257 extending from its proximal first end portion into the locking space 170, the buttress body 250 being pivotable about the living hinge 160 to move into the locking space 170 such that the outer surface 251 of the proximal first end portion of the buttress body abuts against the curved inner surface 243 of the first jaw structure 241 and a ball-shaped member 259 on the distal tip of the projection 257 enters an opening 231 shaped by said clip hinge 230 (thus having a “C-shaped” cross-section) to hold said projection 257 in place by an interference fit, to bias the clip 200 in a closed position, as shown FIG. 16a. As further illustrated in FIG. 17, both projection 257 and ball-shaped member 259 may extend laterally across the entire lateral width of the clip 200, such that ball-shaped member 259 is actually a rounded laterally-spanning cylindrical surface member with fits into a lateral slot formed by opening 231 in clip hinge 230 which also extends laterally across the width of the clip 200. The mating of member 259 in opening 230 causes the parts to lock together thereby preventing the body 250 from moving out of space 170. Like clip 100, in clip 200, once the buttress body 250 is in the “locked” position as shown in FIG. 16a, the first and second jaws 241, 242 are urged or spread apart (shown, as an example, by arrows “J1” and “J2” in FIG. 16) by action of surfaces of the wedge/buttress body 250 acting on portions of curved inner surfaces 243, 244, which act as moments about the clip hinge 230 and lateral pivot axis P to urge the legs 201, 202 and their inner clamping surfaces 231, 232 to become further apposed to each other, thereby providing additional clamping and closing force over a vessel around which the clip 200 is applied. This apposition defines a “fully closed position” of the clamping surfaces 231, 232 as shown in FIG. 16a where no vessel is positioned therebetween. The means for actuating the clip 200 and its proximal locking mechanism are similar to those described above with regard to clip 100, as well as the method by which the clip 200 may be inserted, positioned, opened, and closed using one or more instruments or appliers.

The clip 200 may be in a range of sizes, similar to those of clip 100, and is shown in FIGS. 18, 18a as an embodiment having a particular size in inches as indicated in the figure. An overall length “S21” of the clip 100 may be approximately 0.5 inches; the length “S22”, between the intersection of transverse axis T and longitudinal axis L centered at clip hinge 130 and the distal tip of the clip, may be approximately 0.4 inches, and an overall width “W1” (including the clip hinge 230) may be 0.150 inches while the width “W2” of the clip legs 201, 202 may be approximately 0.100 inches. The illustration of clip 100 in FIG. 18 is shown as a scaled magnification of actual size, and shows all the parts of the clip 200 in actual proportion to each other.

It is further generally understood that the features of clip 100 and clip 200 may be interchanged as further embodiments of the present invention.

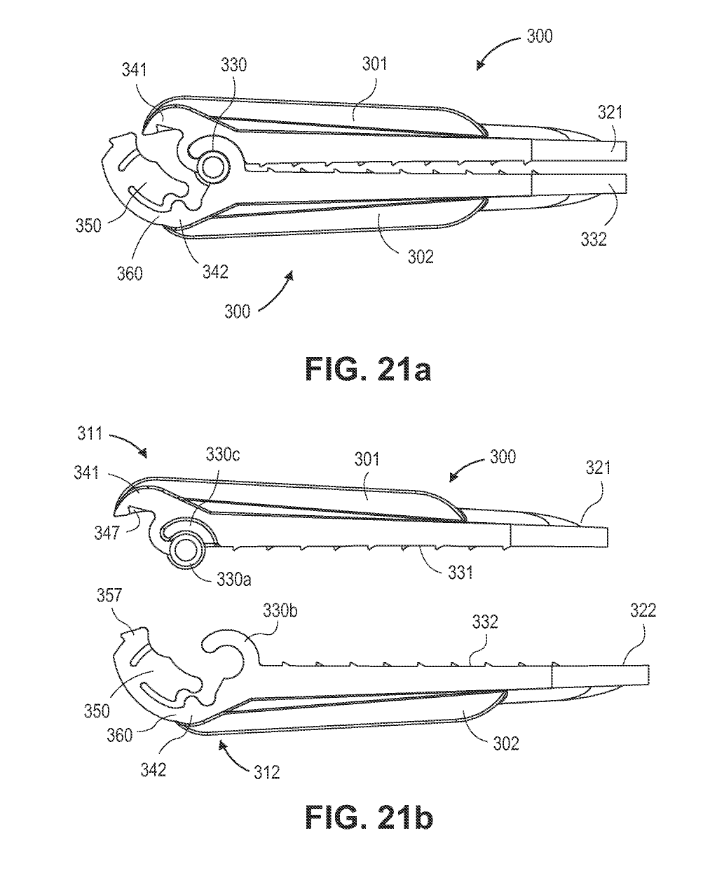

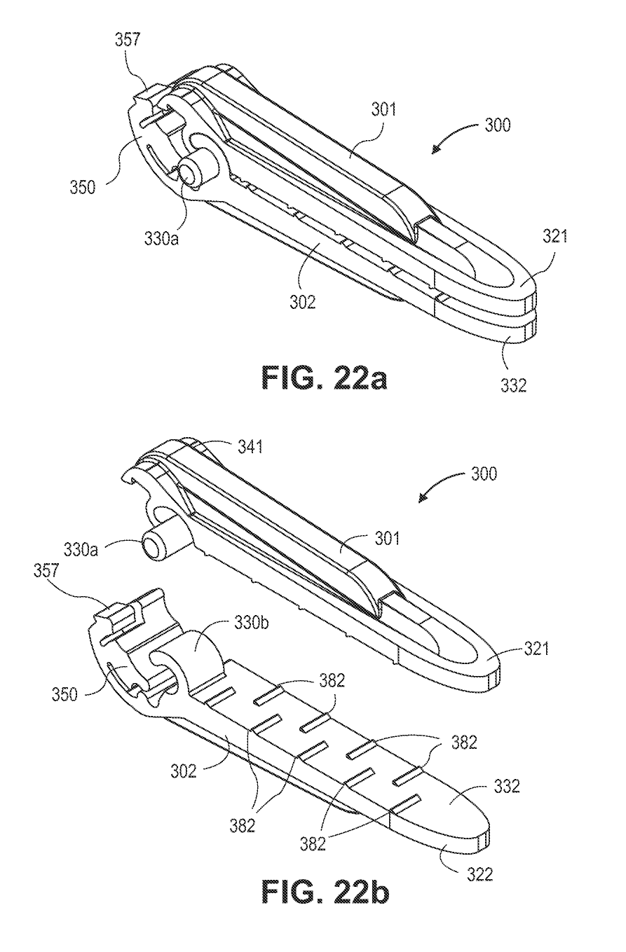

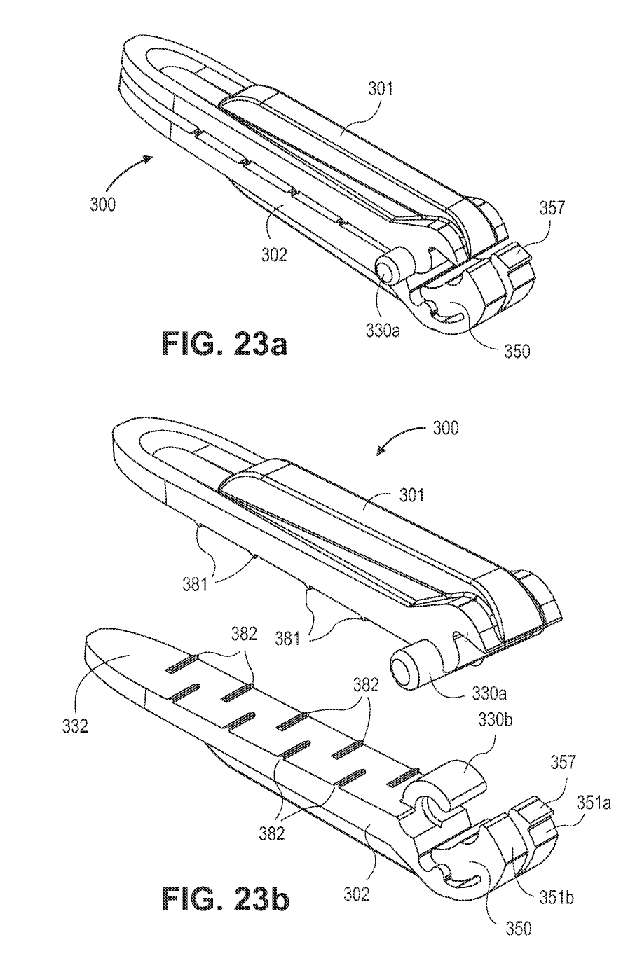

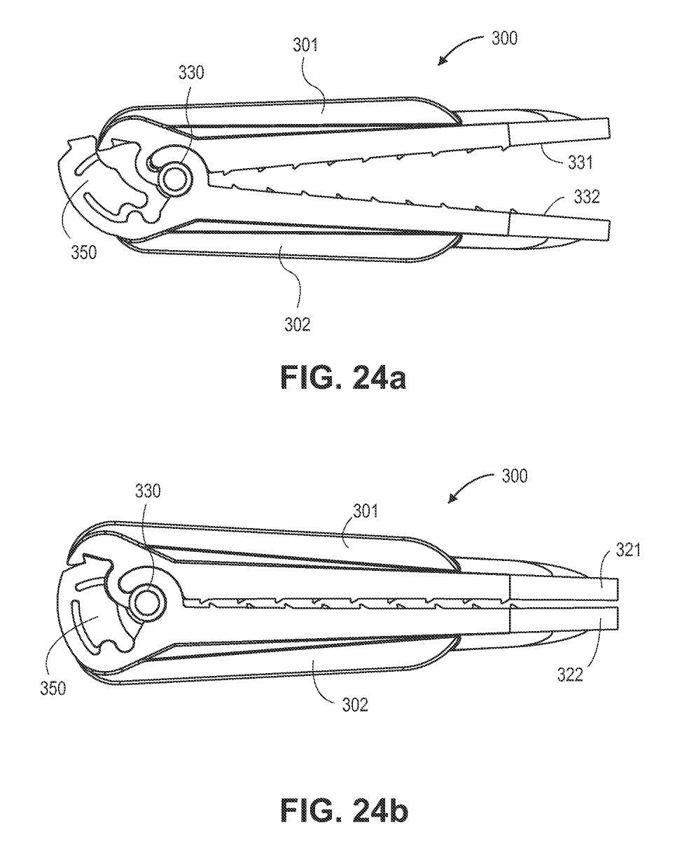

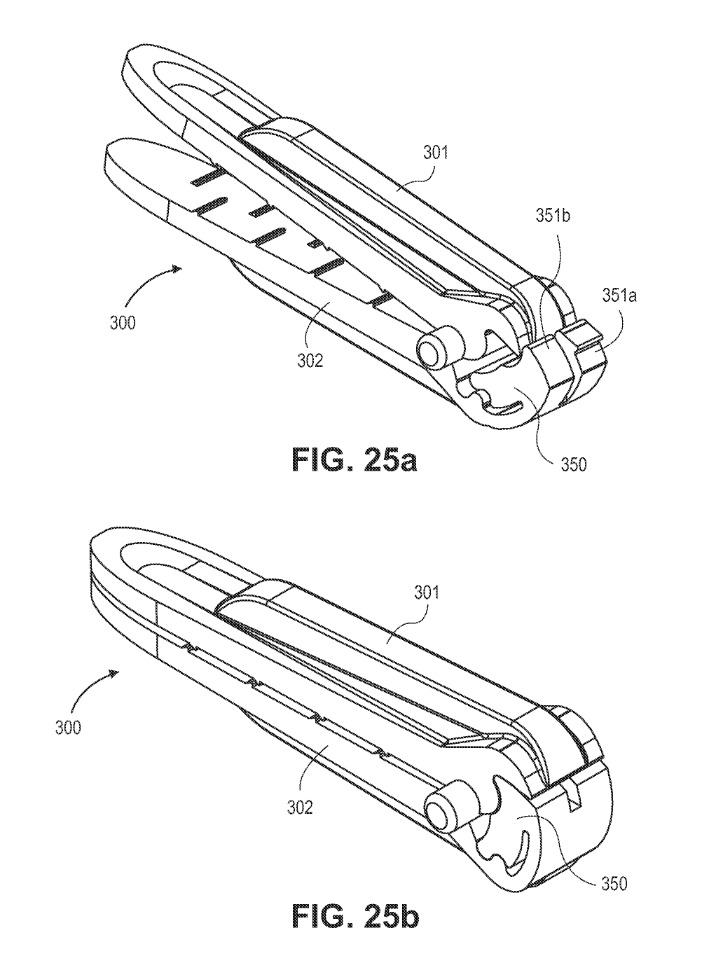

FIG. 21a is a side view of a surgical ligation clip according to another embodiment of the invention, wherein a clip 300 has two parts which are separable, generally being legs 301 and 302. In clip 300, a clip hinge 330 is formed by a separable interconnection pivotally joining the first and second legs 301, 302 at a point on their respective proximal end portions 311, 312, the first leg 301 defining a lateral hinge pivot bar 330a and a laterally spanning curved groove 330c disposed about a portion of the hinge pivot bar 330a, the second leg 302 defining a complementary hook portion 330b configured to mate with the laterally spanning curved groove 330c of the first leg 301. As best shown in FIG. 23b, the hook portion 330b spans laterally only a partial width of clip 300. Clip 300 includes inner clamping surfaces 331, 332 which extend from the hinge interconnection 330 to the distal ends 321, 322 of the legs 301, 302, each clamping surface having teeth 381, 382. Similar to clip 100, proximal jaws 341 and 342 extend proximal of hinge 330, and a buttress body 350 pivots about a living hinge 360 and extending from proximal end of jaw 342 on second leg 302. The proximal locking mechanism of clip 300 is in most respects nearly the same as in clip 100, and includes a notch 347 and detent 357 as well as curved planar segment abutment portions 351a and 351b.

It is further generally understood that the features of clip 100 and clip 300 may be interchanged as further embodiments of the present invention.

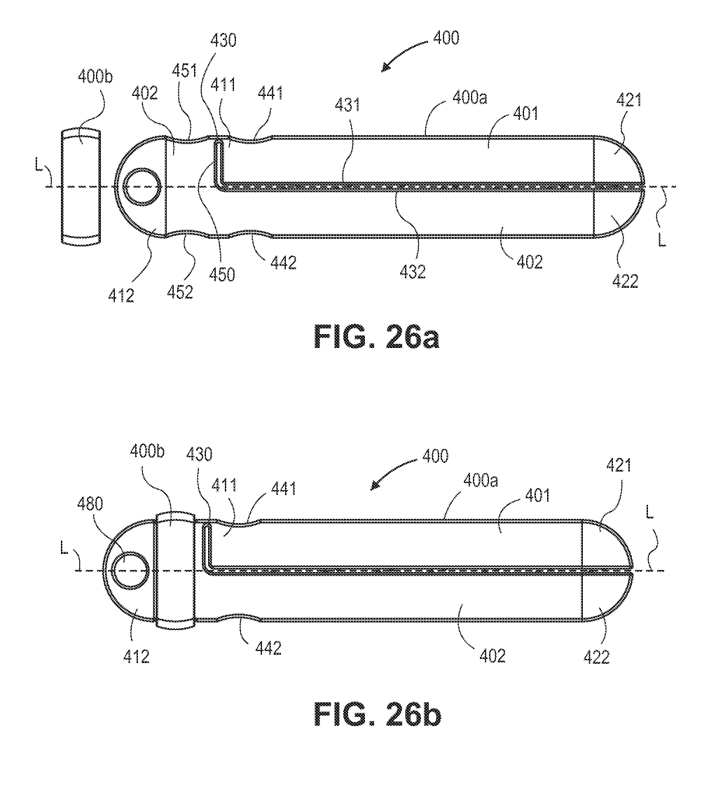

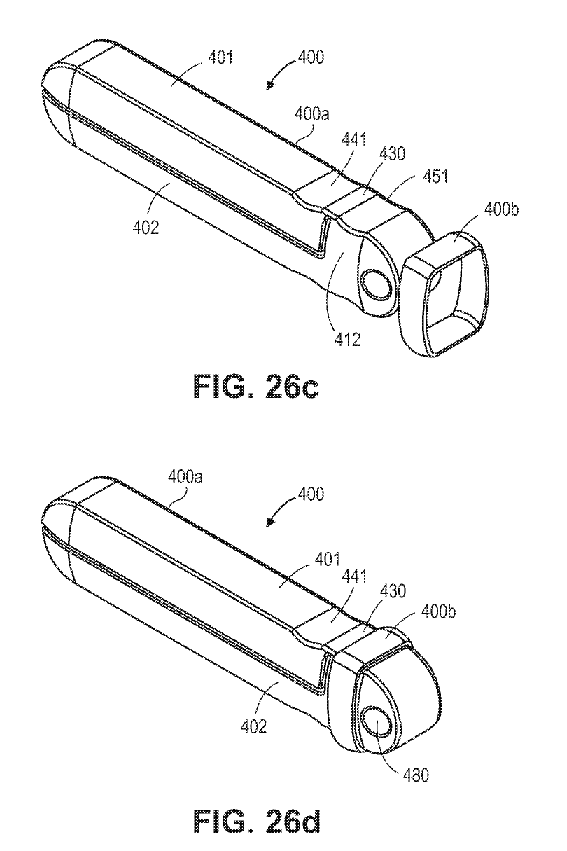

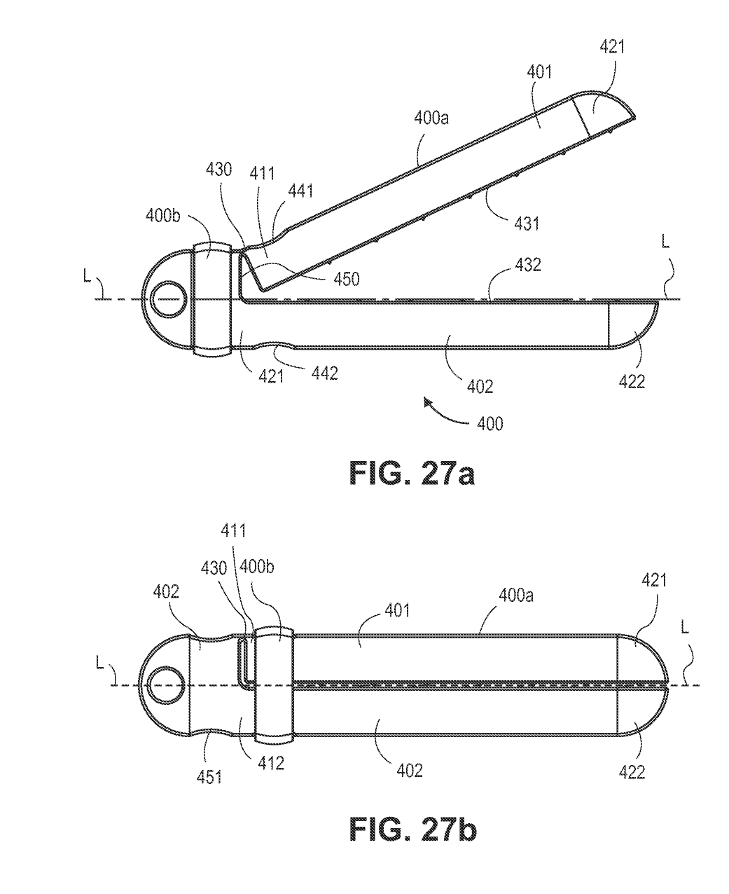

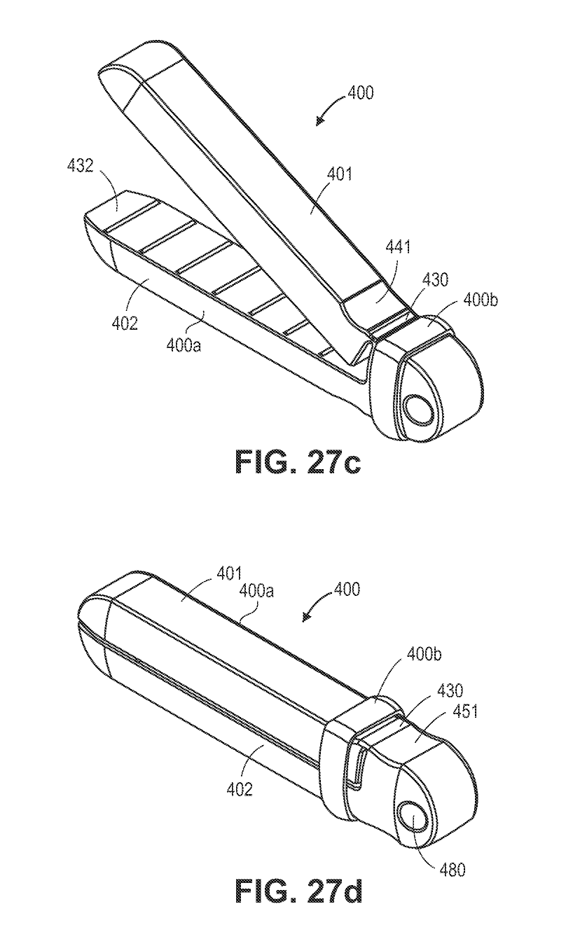

FIG. 26a is a side view of a surgical ligation clip assembly according to another embodiment of the invention. A surgical ligation clip assembly 400 includes a surgical clip 400a defining a longitudinal axis L and having first and second legs 401, 402 each extending along the longitudinal axis and having proximal 411, 412 and distal 421, 422 end portions with respect to said longitudinal axis. The first and second legs 401, 402 each have inner clamping surfaces 431, 432 spanning between the proximal and distal end portions of said first and second legs 401, 402. The inner clamping surfaces 431, 432 are opposed and substantially parallel to the longitudinal axis when the clip is in a legs-closed position, as shown in FIGS. 26b and 27b. A clip hinge 430 joins the first and second legs 401, 402. In the assembled state of clip 400, as shown in FIGS. 26b and 26d, a locking ring 400b is disposed in a first position over proximal indentations 451, 452 around the clip longitudinally proximal to the clip hinge 430, prior to application of the clip 400 around a vessel. The clip 400 defines indentations 441, 442 on respective outer surfaces of the first and second legs 401, 402 immediately distal to the clip hinge for receiving the locking ring 400b. After closing the clip, the locking ring 400b can be moved distally past the hinge 430 and positioned over the indentations 441, 442 to lock the clip 400 in a legs-closed position, as shown in FIGS. 27b and 27d. In one embodiment, the clip hinge 430 joins the second leg 402 at a proximal end of the first leg 401, the clip hinge 430 being transversely offset from the inner clamping surfaces 431, 432 when clip 400 is in the legs-closed position. In the illustrated embodiment, the inner clamping surfaces 431, 432 are disposed between a longitudinal position of the clip hinge and the distal end portions 421, 422 of said first and second legs 401, 402, and the second leg 402 forms the proximal portion of the clip 400 and defines a transverse shoulder 450 extending from the clip hinge 430 to the inner clamping surface 432 of the second leg 402, into which the proximal end 411 of the first leg 401 abuts when the clip 400 is in a legs-closed position. The clip 400 may be moved through an instrument prior to positioning the clip proximate the vessel, wherein a portion of the instrument may also open the clip from a closed position to an open position. A through-hole or pair of side indentations 480 may be formed laterally into the proximal end of the clip 400.

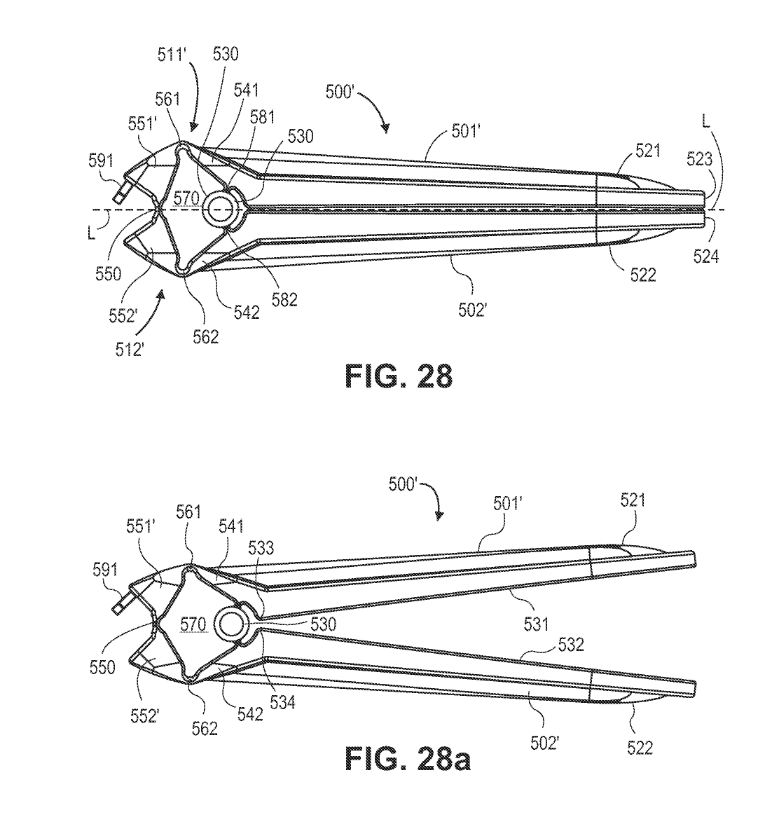

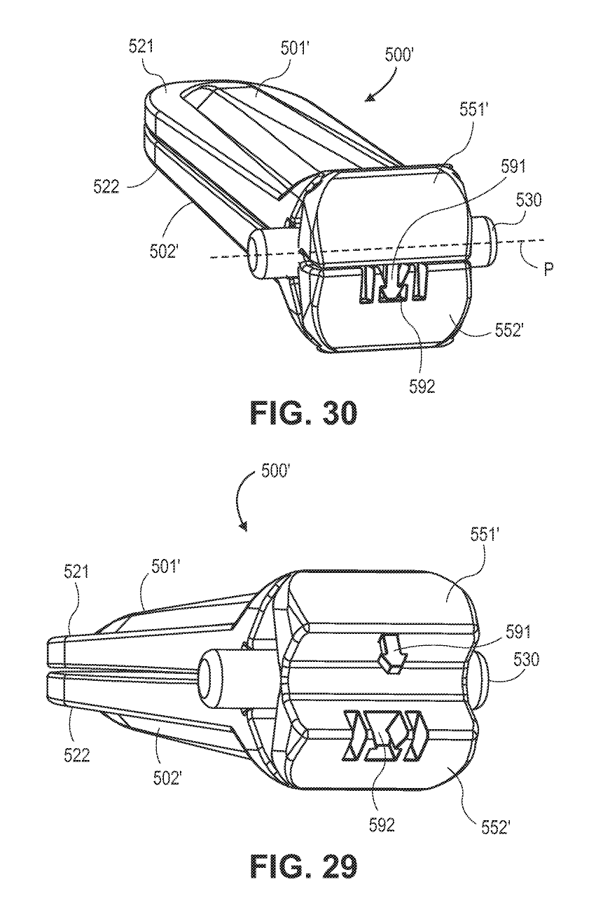

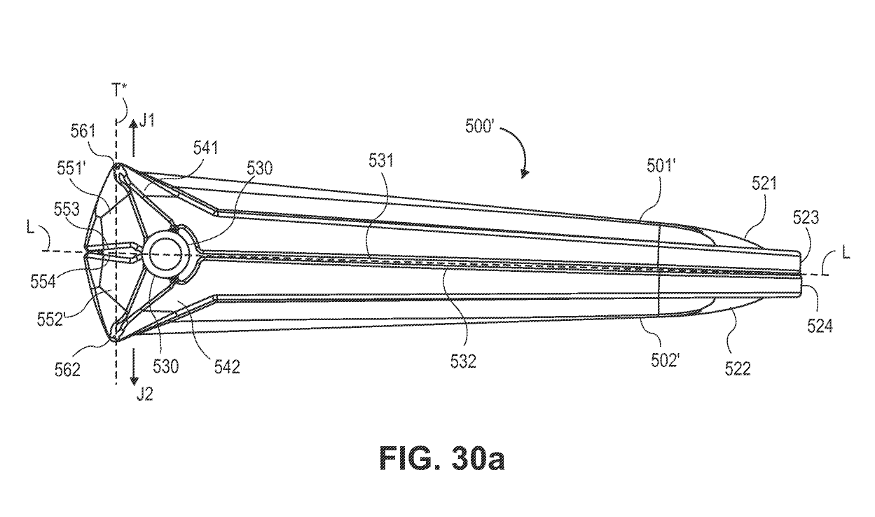

FIG. 28 is a side view of a surgical ligation clip according to another embodiment of the invention. Clip 500′ defines a longitudinal axis “L” along its longest dimension and includes a first leg 501′ and a second leg 502′ each extending along the longitudinal axis L and having proximal 511′, 512′ and distal 521, 522 end portions with respect to said longitudinal axis. A clip hinge 530 joins the first and second legs 501′, 502′ at a point on their respective proximal end portions 511′, 512′, the first and second legs each having respective inner clamping surfaces 531, 532 between the clip hinge 530 and the distal ends 523, 524 of said first and second legs 501′, 502′, the clamping surfaces being apposed when the clip 500′ is in a fully closed position. The clip hinge 530 can include a bar or cylindrically shaped body or tube which defines a lateral pivot axis “P” (shown in FIG. 30) about which the legs 501′ and 502′ pivot as the clip moves from open to a closed position and vice versa. A first jaw structure 541 on the first leg 501′ extends proximal to a transverse axis which is perpendicular to the longitudinal axis L and lateral pivot axis P, centered on the clip hinge 530. The first jaw structure 541 is connected to a first proximal heel portion 551′ by a living hinge 561, the heel 551′ and jaw 541 each being angled to the longitudinal axis L such that the combination of the two elements forms a bent and pivotable moment arm about pivot axis P and clip hinge 530. A second jaw structure 542 is on the second leg 502′ extending proximal to the hinge 530 is connected to a second proximal heel portion 552′ by a living hinge 562, the heel 552′ and jaw 542 each being angled to the longitudinal axis L such that the combination of the two elements forms a bent and pivotable moment arm about pivot axis P and clip hinge 530. The proximal heels 551′ and 552′ are triangular in cross-section and are coupled to each other by an integral living hinge 550.

The portion of the clip 500′ proximal to the hinge 530 thus forms a “spreading section”, while the portion distal to the hinge 530 is a clamping section. As described herein, the surgical clip 500′ of the present invention provides that the jaws 541 and 542 are each substantially proximal to a transverse plane extending through transverse axis and lateral pivot axis P, thus behind the clip hinge 530. The clip hinge 530 can be a resilient hinge providing biasing force to maintain the inner clamping surfaces 531, 532 of the legs 501′, 502′ towards a closed position, without having to lock the distal ends 523, 524 to each other or use a clip applier tool which acts on said distal ends 523, 524, thereby obviating the need to dissect tissue around the distal end of the clip as in previously known surgical ligation clips. However, in the embodiment shown in FIG. 28, the heels 551′ and 552′ are sized such that when they are pressed into a space 570 formed on the proximal end portion of clip 500′ between said jaws 541 and 542 as shown in FIG. 30a, they spread apart said jaws (in the direction of arrows J1 and J2 shown in FIG. 27) and thus act as moment arms about clip hinge 530 and pivot axis P to force the inner clamping surfaces 531, 532 of legs 501, 502 together. To open the legs of the clip 500′, external forces may be applied in the directions opposite to arrows J1 and J2, such as by an applier instrument. Furthermore, the heels 551′ and 552′ are sized and shaped such that when pressed into space 570, they travel “over center” and lock themselves in place in the “spreading section” of the clip 500. As shown in FIG. 30a, distal portions of the heels 551′ and 552′ cross a transverse plane “T*” running through both living hinges 561 and 561. To pivot the heels 551′ and 552′ from the unlocked position shown in FIG. 28 to the locked position shown in FIG. 30a, an external force may be applied to the proximal end of clip 500′ which can be generally parallel to longitudinal axis L or angled toward it. As shown in FIG. 30a, the heel portions 551′ and 552′ are triangular in cross-section and each further define a proximal end outer surface 553, 554 facing the longitudinal axis L when the heels 551′ and 552′ are in a locked position and which mate in partial apposition when the heel portions are pivoted into the locking space to bias the clip in a closed position, as shown in FIG. 30a, and further stabilize the locked position of the proximal portion of the clip 500′ as shown.

Clip 500′ includes complementary interlocking means disposed on the respective proximal end outer surfaces 553, 554 of the proximal heel portions 551′ and 552′. In one embodiment, the first proximal pivoting heel 551′ includes a male engaging feature 591 which fits into a female engaging feature 592 on second proximal heel 552′. This couples the heels to each other so that they hold in place more when pressed into the locked position shown in FIG. 30a. Furthermore, the legs 501′ and 502′ of clip 500′ are coupled to clip hinge 530 by attachment points 581 and 582 which are transversely offset and separated from the longitudinal axis L so as to provide a greater spacing of proximal ends 533 and 534 of inner clamping surfaces 531 and 532 when the clip legs are in the open position as shown in FIG. 28a, which aids in clamping around a larger mass of tissue of a wider vessel.

It is further generally understood that the features of clip 100 and clip 500′ may be interchanged as further embodiments of the present invention.

The clamping surfaces of the clips of the present invention, as disclosed in the embodiments discussed herein, may have clamping surfaces with a concave arc designed in place, such that the proximal and distal ends of the clamping surfaces are in close proximity, while the centers are further apart. The arcing surface will provide a more even distribution of force along the clamping surface when the clip is in locked closed condition.

The instrumentation used to deploy the clips discussed herein may include a manually loaded device that can apply a single clip at a time, or an automatically fed, multiclip applier. Both appliers can be endoscopic instruments suitable for use in laparoscopic surgery applications. In both cases the applier will clamp over the vessel to flatten the section to be ligated. The clip will then be opened, positioned over the vessel and closed. Once closed, a mechanism will engage the locking feature on the proximal end of the clips disclosed herein, to the to maintain the clamping pressure of the clip. A manual applier will load/apply a single clip at a time. An automatic applier will be able to load/apply multiple clips before the instrument has to be removed from the surgical site. The sequence of clip application is as follows:
1. The clip is presented in the partially closed condition.
2. A device, such as a set of applier jaws clamps down on the vessel or tissue to be ligated or clamped. The applier jaws have a channel down the center that is just large enough to allow the clip to fit in the channel.
3. The clip is opened by pressing the proximal legs together lightly.
4. The clip is advanced over the vessel or tissue that is clamped within the jaws of the applier (the clip traveling in the channel area of the applier jaws).
5. Once fully advanced, the proximal legs are released and the clip springs back to the partially closed condition.
6. The proximal locking mechanisms discussed for the clip embodiments disclosed herein are actuated or pressed, causing the legs or ‘clamping section’ of the clips to close tightly on the vessel or tissue.
   The various embodiments of the clips disclosed herein therefore can start in an as-molded state; can be opened further to better encapsulate the vessel; and can then be closed further (into a 3rd state). This process of opening and closing the clip can be repeated as needed, prior to locking. When closed and locked, the clip provides an active clamping force which can also squeeze the vessel, which is beneficial if the vessel necroses and/or shrinks over time.

The various embodiments of the surgical clips of the present invention are preferably made of one or more polymer materials, such as, by example, acetyl homopolymer, but could also be made of a variety of other materials, including one or more metals, or a combination of metal and polymer or plastic. In selecting the material(s) used, the radiopacity of the clip can be “tuned” to a desirable level, or can be tuned to be radiopaque.

The various embodiments of surgical clips of the present invention are an improvement over the known polymeric surgical ligation clips, as well as standard metal clips. Among the resulting advantages of the surgical clip of the invention as disclosed herein are: the ability to deliver a larger clip through a smaller endoscopic instrument; the ability to place a clip on a vessel just like a prior art malleable and deformable metal clip, with no need for added dissection or cleaning around the vessel, but with greater retention force than metal clips, which results in a reduced risk of clips slipping off the vessels. The greater clip locking stability and clip retention force is accomplished by the locking feature applying an active biasing or clamping force as discussed above, versus the passive clamping action created by plastic deformation of malleable metal clips.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention. All ranges cited herein specifically incorporate all values and sub-ranges within the cited range.

What is claimed is:

1. A surgical ligation clip, defining a longitudinal axis and comprising:
first and second legs each extending along the longitudinal axis and having proximal and distal end portions with respect to said longitudinal axis,
a clip hinge joining the first and second legs at a point on their respective proximal end portions, the first and second legs each having inner clamping surfaces between the clip hinge and the distal end portions of said first and second legs, the clamping surfaces being apposed when the clip is in a fully closed position,
a first jaw structure on the first leg extending proximal to the clip hinge, the first jaw structure having a first curved inner surface extending from the clip hinge and facing the longitudinal axis and being substantially concave viewed from said axis,
a second jaw structure on the second leg extending proximal to the clip hinge and having a second curved inner surface extending from the clip hinge, and
a buttress body extending from and connected to the second jaw structure by a first living hinge at a proximal end of said second jaw structure, the buttress body having an outer surface on a proximal first end portion thereof on a proximal end of the clip,
the first and second jaw structures being spaced from the longitudinal axis on opposite sides thereof and defining a locking space therebetween, the buttress body being pivotable about the first living hinge to move into the locking space such that a curved planar segment abutment portion of the outer surface of the proximal first end portion of the buttress body abuts against the curved inner surface of the first jaw structure to bias the clip in a closed position.

2. The surgical clip of claim 1, wherein a proximal end portion of the curved inner surface of the first jaw structure defines a notch recessed from said curved inner surface, and the buttress body defines a detent formed on the outer surface thereof, the detent mating with the notch when the buttress body is pivoted into the locking space to bias the clip closed in the closed position.

3. The surgical clip of claim 2, wherein the notch and detent are formed on corresponding partial lateral sections of the buttress body and first jaw structure, respectively.

4. The surgical clip of claim 3, wherein a first partial lateral section of the buttress body has a transverse width sufficient to exceed a complementary width formed by the locking space to create an interference fit between the proximal end portion of the curved inner surface of the first jaw structure and a portion of the curved planar segment abutment portion of the outer surface on the proximal first end portion of the buttress body, and the detent is formed on a second partial lateral section of the buttress body distinct from the first partial lateral section.

5. The surgical clip of claim 4, wherein the buttress body defines a second living hinge between the proximal first end portion thereof and a distal second end portion, and wherein a proximal first end portion of the second partial lateral section of the buttress body is capable of pivoting about said second living hinge when the buttress body moves into the locking space, allowing a lateral sectional portion of the curved planar segment abutment portion of the outer surface of the proximal first end portion of the buttress body to flex towards the longitudinal axis prior to abutment against the curved inner surface of the first jaw structure, the lateral sectional portion including the detent.

6. The surgical clip of claim 1, wherein the outer surface of the buttress body defines a laterally spanning notch on the proximal end of the clip and further defines a laterally spanning flange extending from said notch adjacent to the curved planar segment abutment portion.

7. The surgical clip of claim 1, wherein the buttress body occupies a majority of a volume of said locking space in said closed position.

8. The surgical clip of claim 1, wherein the second curved inner surface on the second jaw structure forms a first laterally spanning recessed groove separated from the clip hinge and a first laterally spanning rounded protruding surface proximal to said first recessed groove, and a distal second end portion of the buttress body forms a second laterally spanning recessed groove and a second laterally spanning rounded protruding surface distal to said second recessed groove which are shaped complementary to the first rounded protruding surface and first recessed groove, respectively, so as to mate in abutment when the buttress body is pivoted into the locking space to further stabilize and bias the clip in said closed position.

9. The surgical clip of claim 1, wherein an overall longitudinal length of the clip is in a range from approximately 0.15 inches to approximately 0.5 inches.

10. The surgical clip of claim 1, wherein the clip is made entirely of a polymer material.

11. The surgical clip of claim 1, wherein each of the inner clamping surfaces define a plurality of teeth and grooves, the teeth and grooves on the first leg being aligned complementary to grooves and teeth, respectively, of the second leg.

12. The surgical clip of claim 1, wherein each of the inner clamping surfaces further defines a concave radius of curvature when facing transversely away from the longitudinal axis towards said inner clamping surface.

13. The surgical clip of claim 1, wherein the clip hinge is a separable interconnection pivotally joining the first and second legs at a point on their respective proximal end portions, the first leg defining a lateral hinge pivot bar and a laterally spanning curved groove disposed about a portion of the hinge pivot bar, the second leg defining a complementary hook portion configured to mate with the laterally spanning curved groove of the first leg.

14. The surgical clip of claim 1, wherein the clip hinge is formed by a laterally extending bar integrally formed with the first and second legs, each leg being resiliently coupled to first and second transverse sides of said bar, the bar further defining laterally spanning grooves on longitudinally proximal and distal sides of the bar.

15. The surgical clip of claim 1, further comprising flanges extending longitudinally across respective outer surfaces of each of the first and second legs which are on opposite sides to the inner clamping surfaces of each respective leg, the flange of the first leg extending from the first jaw structure to the distal end portion of the first leg, the flange of the second leg extending from the second jaw structure to the distal end portion of the second leg.

16. The surgical clip of claim 15, wherein each of the flanges defines a transverse indentation proximate the distal end portions of the legs.

17. The surgical clip of claim 1, wherein the curved planar segment abutment portion of the outer surface of the proximal first end portion of the buttress body defines a lateral span equal to at least the width of each of the legs.

18. A surgical ligation clip, defining a longitudinal axis and comprising:
first and second leg means each extending along the longitudinal axis and having proximal and distal end portions with respect to said longitudinal axis, clip hinge means joining the first and second leg means on respective proximal end portions thereof, the first and second leg means each having inner clamping surface means between the clip hinge means and the distal end portions of said first and second leg means, the clamping surface means being apposed when the clip is in a fully closed position,
locking means for biasing the legs closed, said locking means extending proximal to the clip hinge means, the locking means being capable of actuation by application of an external force substantially along said longitudinal axis to a proximal end of the clip which forms an integral portion of said locking means, to move at least one body disposed on the second leg means as a first part of said locking means from a first position to a second position to provide an abutment force between a curved planar surface segment or segments on said body and a complementary surface formed on a second part of said locking means disposed on the first leg means to bias the clip in a closed position.

19. A method of applying a surgical ligation clip, comprising:
positioning the clip in an open position proximate an inner anatomical body vessel, the clip having
first and second legs each extending along a longitudinal axis of the clip and having proximal and distal end portions with respect to said longitudinal axis,
a clip hinge means joining the first and second legs at a point on their respective proximal end portions, the first and second legs each having inner clamping surface means between the clip hinge and the distal end portions of said first and second legs, the clamping surface means being apposed when the clip is in a fully closed position,
a locking means for biasing the legs closed extending proximal to the clip hinge means, and
applying an external force substantially along the longitudinal axis to a proximal end portion of one of the legs which forms a portion of the locking means, to move a body formed as a first part of said locking means from a first position to a second position to provide an abutment force between a curved planar segment abutment portion of said body and a curved surface formed on a second part of said locking means disposed on the first leg to bias the clip in a closed position.

20. The method of claim 19, further comprising moving the clip through an instrument prior to positioning the clip proximate the vessel, wherein a portion of the instrument opens the clip from a closed position to an open position.
